(19) United States
(12) Patent Application Publication
(10) Pub. No.: US 2013/0281548 A1
(43) Pub. Date: Oct. 24, 2013

(54) COMPOSITIONS COMPRISING A COMBINATION OF AT LEAST ONE COLORANT AND AT LEAST ONE POLYSACCHARIDE

(71) Applicant: EPC (BEIJING) NATURAL PRODUCTS CO., LTD., Beijing (CN)

(72) Inventors: Zhijin Jin, Beijing (CN); Jingang Shi, Beijing (CN)

(73) Assignee: EPC (Beijing) Natural Products Co., Ltd., Beijing (CN)

(21) Appl. No.: 13/840,005

(22) Filed: Mar. 15, 2013

Related U.S. Application Data
(60) Provisional application No. 61/635,594, filed on Apr. 19, 2012.
(60) Provisional application No. 61/783,578, filed on Mar. 14, 2013.

(51) Int. Cl. A23L 1/275; A61K 47/36; A61K 8/73

(52) U.S. Cl. CPC A23L 1/2751; A61K 8/73; A61K 47/36
USPC 514/777; 426/250; 426/540; 106/501.1

(57) ABSTRACT

Dispersible colorants that include a combination of a colorant and a polysaccharide are provided, wherein the weight ratio of total colorant to total polysaccharide is in the range of 5000:1 to 1:5000. In one aspect, the colorant is a synthetic or natural colorant. In one aspect, polysaccharide is interpreted very broadly and provides a colored precipitate upon the combination of the colorant with the polysaccharide.

### CROSS REFERENCE TO RELATED APPLICATIONS

This application claims priority to U.S. Provisional Patent Application No. 61/635,594, filed Apr. 19, 2012, and U.S. Provisional Patent Application No. 61/783,578, filed Mar. 14, 2013 all entitled “COMPOSITIONS COMPRISING A COMBINATION OF AT LEAST ONE COLORANT AND AT LEAST ONE POLYSACCHARIDE”, the contents of which are incorporated herein in their entirety for all purposes.

### FIELD OF THE INVENTION

Dispersible colorants comprising a combination of a colorant and a polysaccharide are described. In particular, dispersible colorants comprising a combination of a colorant and a polysaccharide are provided that are useful in any applications wherein conventional colorants, including pigments, lakes, etc., can be applied, and are especially, useful in the manufacture of food products, sweets, cosmetics, toys, pharmaceutical products, etc.

### BACKGROUND OF THE INVENTION

Colorants are materials that change the color of reflected or transmitted light as the result of wavelength-selective absorption. Conventionally, colorants are widely used for coloring paint, inks, plastics, fabrics, cosmetics, foods and other materials.

A problem in the application of colorants is that many colorants, especially natural colorants, are more or less water soluble. They will leak and “bleed” (i.e., diffusion of color from the substrate into the environment) if in an aqueous environment, which is a problem in many applications.

Another problem in the application of colorants is that many colorants, especially natural colorants, have poor thermal/light stabilities. This is problematic in many applications, such as, in the manufacture of food products, sweets, cosmetics, toys, pharmaceutical products, etc.

To overcome the above mentioned problems of colorants, lakes have been developed. Lakes are color additives prepared by precipitating a soluble colorant onto an acceptable insoluble base or substrate. The substrates can include, but are not limited to, alumina, zinc oxide, calcium carbonate or talc. Lakes impart color by being dispersed into a medium. When compared to pure colorants, lakes have superior opacity and improved thermal/light stability. Among others, aluminum lakes are conventionally applied due to their superior performances in comparison with lakes based on other substrates.

Aluminum is the third most abundant element, and the most abundant metal, in the Earth's crust. The effects of aluminum on humans have been extensively reviewed. Despite such studies, there is little indication that aluminum is acutely toxic for the general population, its health effects are of interest in view of the widespread occurrence of the element in the environment and in commerce.

Literature has reported that prolonged exposure to aluminum can cause systemic toxicity, mainly affecting the gastrointestinal tract and causing neurological and skeletal effects. Some toxicity can be traced to deposition in bone and the central nervous system, which can be increased in patients with reduced renal function. Because aluminum competes with calcium for physiological absorption, increased amounts of dietary aluminum may contribute to reduced skeletal mineralization (osteopenia) observed in preterm infants and infants with growth retardation.

In very high doses, aluminum can cause neurotoxicity, and can be associated with altered function of the blood-brain barrier. A small percentage of people are allergic to aluminum and experience contact dermatitis, digestive disorders, vomiting or other symptoms upon contact or ingestion of products containing aluminum, such as deodorants or antacids. In those without allergies, aluminum is not as toxic as heavy metals, but there is evidence of some toxicity if it is consumed in excessive amounts. Furthermore, aluminum salts increase estrogen-related gene expression in human breast cancer cells cultured in the laboratory. The estrogen-like effects of these salts have led to their classification as a metalloestrogen.

The occurrence of aluminum in antiperspirants, or colorants, such as aluminum lakes, and/or food additives is controversial in some scientific quarters. Aluminum in food may be absorbed more effectively than aluminum from water. Some researchers have expressed concerns that aluminum in antiperspirants may increase the risk of breast cancer, and aluminum has controversially been implicated as a factor in Alzheimer's disease. According to The Alzheimer's Society, the overwhelming medical and scientific opinion is that studies have not convincingly demonstrated a causal relationship between aluminum and Alzheimer's disease. Nevertheless, some studies, such as those on the PAQUID cohort, cite aluminum exposure as a risk factor for Alzheimer's disease.

In view of the above mentioned toxicity of aluminum, many countries and organizations have decided to establish a maximum limit for aluminum in food additives, especially colorants.

Therefore, a need exists for an alternative colorant for aluminum lakes which have similar or superior performances in comparison with aluminum lakes but overcome one or more of the disadvantages of aluminum-containing lakes, such as toxicity.

### BRIEF SUMMARY OF THE INVENTION

Dispersible colorants are provided that include a combination of a colorant and a polysaccharide, wherein the weight ratio of total colorant to total polysaccharide is in the range of 5000:1 to 1:5000. In one aspect, the colorant is interpreted very broadly and includes synthetic or natural colorants. In one aspect, polysaccharide is interpreted very broadly and provides a colored precipitate upon the combination of the colorant(s) with the polysaccharide.

The dispersible colorants can be the product(s) of absorption, adsorption, complexation, chelation, hydrogen bonding, van der Walls interactions, interpentration, electrostatic interations, hydrophilic/hydrophilic, hydrophobic/hydrophobic, hydrophilic/hydrophobic domain interactions, coating, encapsulation, etc. between the colorant and the polysaccharide. Generally the dispersible colorants precipitate from the reaction mixture to provide a solid material that can be further processed.

In one aspect, an all-natural process and resultant compositions are provided to make water-soluble colors become water insoluble that can be used in lipophilic environments. For example, methods and compositions provided herein produce water-soluble insoluble from water-soluble colors (such as sweet potato red, safflower yellow) that are usable in liphophilic fatty/solid conditions. It is generally known that water-soluble colors can't be used in lipophilic/fatty environments. Although emulsifying technology has been used in some areas, surfactants are essential to obtain a stable matrix. The present technology provided herein avoids the need for surfactants or emulsions to provide a stable solution of a water-soluble color in a fatty or lipophilic environment or in solid matrices.

It has also been discovered that treating the dispersible colorants described herein in a second subsequent step, in a solvent or as a solid, at an elevated temperature of greater than about 85° C. to about 150° C., e.g., 85° C. to about 120° C. boiling or reflux (depending upon the boiling point of the solvent) imparts further advantageous characteristics to the dispersible colorant isolated from the treatment. Alternatively, the dispersible colorant can be subjected to a heated environment, with or without moisture, to a temperature below the melting or degradation point of the dispersible colorant, e.g., from about 85° C. to about 200° C., e.g., from about 85° C. to about 150° C., or from about 85° C. to about 120° C. Not to be limited by theory, it is believed that the subsequent step at elevated temperatures gelatinizes the dispersible colorant to provide a heat treated dispersible colorant. The product is oil soluble. In one aspect, the polysaccharide is a starch, starch derivative or a cross-linked starch.

Not only extending the application areas of water-soluble colors to different environment, such as lipophilic environments, embodiments described herein improve the performance of colors, including natural colors, synthetic colors, water-soluble colors and oil-soluble colors, in terms of stability, dispersibility, homogeneity, lack of bleeding, coloring performance (the ability or suitability of coloring a substance), brilliant colors, reduced or no fading of color after cooking or baking, and elimination or reduction of odors generally associated with water-soluble colorants, etc.

The dispersible colorants described herein can also be applied in many fields that conventional colors can be applied, such as in foodstuffs, medicines, pigments, paints, cosmetics, dyes, etc. In particular, particular dispersible colorants, the combination of natural colors and edible carriers, complies with the regulatory provisions in many countries, and can be used in many areas, such as foodstuffs, medicines, etc.

### BRIEF DESCRIPTION OF THE FIGURES

The patent or application file contains at least one drawing executed in color. Copies of this patent or patent application publication with color drawing(s) will be provided by the Office upon request and payment of the necessary fee.

FIG. 1 shows a dispersible Sweet Potato Red Powder colorant in peanut oil in comparison to sweet potato red powder.

FIG. 2 shows synthetic and natural colors mixed with sunflower oil as well as a dispersible colorant at 0 hours.

FIG. 3 is an enlarged view of FIG. 2.

FIG. 4 is an enlarged view of FIG. 2.

FIG. 5 shows synthetic and natural colors mixed with sunflower oil as well as a dispersible colorant at 24 hours.

FIG. 6 shows that after 24 hours, CD-SP and CD-S were still well dispersed in sunflower oil, with little precipitation and the systems kept their original color, while conventional synthetic and natural colors were substantially precipitated from sunflower oil and the systems became transparent.

FIG. 7 provides that a homogeneous colored system can be obtained with SP-S and CD-SP, but cannot be obtained with SP.

FIG. 8 provides that after 1 hour, the colorant precipitated from the SP-S system, and the color of the SP-S system became lighter, while CD-SP system kept its original color.

FIG. 9 provides that after 15 hours, the colorants were substantially precipitated from the SP system and the SP-S system, and such systems became colorless, however, little colorant precipitated from the CD-SP colorant.

FIG. 10 provides at 0 hours that a homogeneous colored system can be obtained with AB-S and CD-AB, but cannot be obtained with AB.

FIG. 11 provides that after 1 hour, the colorant precipitated from the AB-S system, and the color of the SP-S system changed, while CD-AB system kept its original.

FIG. 12 provides that after 15 hours, the colorants were substantially precipitated from the AB system and the AB-S system, and the color of such systems changed significantly, however, little colorant precipitated from the CD-AB colorant.

FIG. 13 provides various food stuffs with dispersible colorants as described herein.

### DESCRIPTION OF THE INVENTION

Embodiments surprisingly provide a colorant having excellent performance characteristics such as high thermal/light stability, high dispersibility, no bleeding, no toxicity, etc. by combining a colorant with a polysaccharide. The combination of the two can be considered to be gelatinized.

Described herein are dispersible colorants that are all-natural. Water soluble colorants are treated to become water insoluble that are useful in fatty, lipophilic, non-aqueous environments. The dispersible colorants described herein are both water insoluble as well as oil insoluble. The dispersible colorants described herein can be well dispersed in both aqueous as well as oil (lipophilic) environments. Therefore, the unique materials described herein can be considered as “dispersible colorants”, “disperse colorants” or “disperse dyes”, as they are akin to the class of water-insoluble dyes dispersed in aqueous solution in the dyeing of synthetic textile fibers. In one aspect, the colorant is an anthocyanin. In another aspect, the combination is a natural color, such as an anthocyanin and an edible carrier, such as a starch.

Additionally, the dispersible colorants described herein meet the provisions of article 31 of the EC regulations 1907/2006.

Therefore, in a broad aspect, a dispersible colorant is provided comprising a combination of a colorant and a polysaccharide, which is useful in applications wherein conventional colorants, such as pigments and lakes, are typically applied. Specially the dispersible colorants described herein are useful in the manufacture of food products, sweets, cosmetics, toys, pharmaceutical products, etc.

The weight ratio of total colorant(s) to total polysaccharides in the dispersible colorants described herein are not specifically restricted, provided that a stable combination can be formed. In one aspect, the weight ratio is in a range of 1:5000-5000:1, particularly in the range of 1:1000-1000:1, more particularly in the range of 1:100-100:1, most particularly in the range of 1:10-10:1, for example, 1:5000, 1:500, 1:200, 1:100, 1:50, 1:10, 1:1, 10:1, 50:1, 100:1, 200:1, 500:1, 1000:1, and 5000:1 of colorant to polysaccharide.

As used herein, the term “colorant” refers to any material that changes the color of reflected or transmitted light as a result of wavelength-selective absorption. Such colorants can be classified into synthetic colorants and natural colorants, pigments, paints, dyes, inks, etc. according to the source.

Synthetic colorants include, but are not limited to, water-soluble or oil-soluble colorants. Water soluble colorants include those such as amaranth, sunset yellow, erythrosine, tartrazine, new red, indigo, and brilliant blue. Oil-soluble colorants include those such as sudan III.

Natural colorants include, but are not limited to: carotenoids, such as Red Chili Color, Annatto, Gardenia Yellow, etc.; flavonoids, such as Beefsteak Plant Color, Corn Color, Safflower Yellow, Red Cabbage Color, Red Sweet Potato Color, Purple Carrot Color, Elderberry Color, Aronia Color, Red Raddish Color, Grape Skin Color, Bilberry Color, Blackcurrant Color, etc.; quinone colorants, such as cochineal colorants, carminic acid, Lac Dye, etc.; porphyrin colorants, such as Chlorophyll, etc.; betacyanines, such as betalain, etc.; dione colorants, such as curcumin, etc.; monascus colorants, such as monascorubrin, etc.; caramel colorants, such as caramel, etc.; gardenia colorants, such as gardenia blue, etc.; and phycocyanin colorants, such as Spirulina Blue Color, Anthoblue; sodium copper chlorophyllin; beet root red; melanin; paprika red; lutein; β-carotene; lycopene, etc.

An extract, concentrate, or juice from a plant or an animal can also be utilized as the natural colorant according to one aspect described herein. The extract, concentrate, or juice from a plant, useful in the dispersible colorants described herein, include, but are not limited to, those from saffron, safflower, gardenia, bilberry, sweet potato, red cabbage, carrot, grape, marigold, sesame, beet root, paprika, carrot, tomato, etc.

The term “extract” is intended to mean materials obtained from plant sources, such as leaves, twigs, bark, roots, stem, seeds, flowers, berries, fruit, juice, for example, by routine isolation methods from suitable plants sources noted, but not limited to, those described herein. There are various methods for the extraction of, in particuarl, anthocyanins known to those of skill in the art. Some of these methods are described in, for example, U.S. Pat. No. 5,817,354;U.S. Pat. No. 5,200,186; U.S. Pat. No. 5,912,363; U.S. Pat. No. 4,211,577; U.S. Pat. No. 4,302,200 (each incorporated herein by reference).

Examples of suitable anthocyanin-containing plants include, but are not limited to, fruits, vegetables, flowers and other plants selected from the group consisting of Acer macrophyllum, Acer platanoides, acerola, Ajuga reptans, apple, apricot, Artict bramble, avocado, banana, barberry, barley, Begonia semperfiorens, Bellis perennis, Bletilla striata, bilberry, black beans, black soybeans, black, blue and purple potatoes, blackberry, blueberry, bog whortleberry, boysenberry, buckwheat, cacao, Camellia sinensis, canarygrass, Caucasian blueberry, Chimonanthus praecox, celery, Cerasus avium, cherry, cherry laurel, chicory, chive, chokeberry, Cornelian cherry, cornflower, cotoneaster, cowberry, cranberry, crowberry, chrysanthemum, Cynomorium coccineum, Dahlia variabilis, danewort, deerberry, Dendrobium, dwarf dogwood, Echinacea purpea, eggplant, elderberry, fababean, Fatsia japonica, feijoa, fig, garlic, gerbera, ginseng, Globe artichoke, gooseberry, grapes, guava, hawthorn, hibiscus or roselle, Hibiscus Sabdaiffa, highbush blueberry, hollyhock, honeysuckle, Ipomoea purpurea, Iris ensata, Java plum, Jerusalem artichoke, kokum, Laeliocattleya, lentil, loganberry, lupine, lychee, maize, mango, mangosteen, maqui, Matthiola incana, meconopsis, Metrosideros excelsa, millet, mountain ash berry, mulberry, myrtle berry, olive, onion, orange, ornamental cherry, passion fruit, pea, peach, peanut, pear, perilla, petunia, Phalaenopsis, Phalsa, Pharbitis, Pineapple, pistachio, plum, pomegranate, Phragmites australis, purple carrot, quince, rabbiteye blueberry, radish, red and black currant, red and black raspberry, red cabbage, rice, rhubarb, rosehip, rye, saffron, sarracenia, sheepberry, Sophronitis coccinea, sorghum, sparkleberry, strawberry, Fragada Vesca, sugarcane, sunflower, sweet cherry, sweet potato, tamarillo, tamarind, taro, tart cherry, Tulip greigii, turnip, water lily, Weigela, wheat, wild rice, Verbena hybrida, yam and mixtures thereof.

Although there are literally thousands of anthocyanin extracts, all of which should be considered included within the realm of this specification, suitable examples of anthocyanin extracts of particular interest include bilberry extract, blackcurrant extract, cranberry extract, black soybean extract, cowberry extract, blueberry extract and mixtures of two or more thereof.

Typically the extract is concentrated by various methods to provide a solution enriched in anthocyanins. For example, ultrafiltration can be used to remove unwanted components by molecular weight cut offs. The retentate from the filtration can be stored as a liquid or, for example, can then be further concentrated into a powder by spray drying, freeze drying, flash drying, fluidized bed drying, ring drying, tray drying, vacuum drying, radio frequency drying or microwave drying. Ultimately, the extract should contain at least 10% by weight anthocyanin content. Commercially available anthocyanins can be obtained from sources such as Artemis International, Fort Wayne, Ind. Commercially obtained anthocyanin extracts should contain at least 10% by weight anthocyanin content. The extracts, therefore, contain anthocyanin(s) and other plant materials such as other flavinoids, sugars, etc.

Anthocyanin extracts can be further purified by one or more methods known in the art, such as chromatography, gel chromatography, high performance liquid chromatography, crystallization, affinity chromatography, partition chromatography and the like. Identification of the particular anthocyanin(s) can be accomplished by methods know to those skilled in the art and include ¹H NMR, chemical degradation, chromatography and spectroscopy, especially homo- and heteronuclear two-dimensional NMR techniques for the characterization of the isolated anthocyanin compounds.

The term “purified” or “isolated” is used in reference to the purification and/or isolation of one or more anthocyanins from an anthocyanin extract as described above. Again using conventional methods known in the art, various components of the anthocyanin extract can be separated into purified materials. In one aspect, the anthocyanin(s) of the extract are substantially purified and isolated by techniques known in the art. The purity of the purified compounds is generally at least about 90%, particularly at least about 95%, and most particularly at least about 99% and even more particularly at least about 99.9% (e.g. about 100%) by weight.

The anthocyanin extract contains one or more anthocyanins and/or anthocyanidins selected from the group consisting of peonidin, cyanidin, pelargonidin, delphinldin, petunidin, malvidin, apigenindin, auratinidin, capensinidin, europinidin, hirsutidin, 6-hydroxycyanidin, luteolinidin, 5-methylcyanidin, pulchellidin, rosinidin, tricetnidin, derivatives and mixtures thereof. In one embodiment, the anthocyanins and anthocyanidins are selected from the group consisting of cyanidin, peonidin, malvidin, petunidin, delphinidin, their glycoside derivatives, and mixtures thereof. In yet another embodiment, the extract contains at least one cyanidin-based anthocyanin.

Anthocyanins that can be useful in the dispersible colorants described herein include, but are not limited to, cyanidin-3-glucoside; cyanidin 3-glucosylrutinoside; cyanidin-3-gentibioside; cyanidin-3-rutinoside, cyanidin-3-sambunigrin, cyanidin-3-samb-5-glucoside, cyanidin-3-galactoside, peonidin-3-rutinoside, peonidin-3-glucoside, peonidin-3-galactoside, peonidin, cyanidin, cyanidin-3 sophoroside, pelargonidin, delphinidin, delphinidin-3-glucoside, delphinidin-3-galactoside, petunidin, petunidin-3-glucoside, petunidin-3-galactoside, malvidin, malvidin-3-arabinoside, malvidin-3-glucoside, malvidin-3-galactoside, kaempferol, hesperidin, gentiodelphin, platyconin, cinerarin and the like.

Suitable examples of anthocyanins from various plants, include, but are not limited to Acer macrophyllum, Cyanidin derivative, Acer platanoides, Cyanidin 3-(2″,3″-digalloyl-beta-glucopyranose (3%), Cyanidin 3-(2″-galloyl-beta-glucopyranose (37%), Cyanidin 3-beta-glucopyranoside (60%), Acerola, Malpighia marginata, Cyanidin-3-glucoside, Cyanidin-3-glucoside, Ajuga reptans, Cyanidin 3-(di-p-coumaroyl) sophoroside-5-glucoside, Apple, Malus spp, Cyanidin 3-galactoside, Cyanidin 3-galactoside, Cyanidin 3-arabinoside, Cyanidin 3-glucoside, Cyanidin 3arabinoside, Cyanidin 3-xyloside, Cyanidin 3glucoside, Cyanidin 3-xyloside, Apricot, Prunus armeniaca, Cyanidin-3-glucoside, Cyanidin-3glucoside, Artic bramble, Rebus spp, Avocado, Persea spp, Acylated cyanidin 3,5-diglucoside, Cyanidin 3-galactoside, Cyanidin 3-galactoside, Banana, Musa acuminata, M. balbisiana, Barberry, Berberis spp., Cyanidin-glucoside, Cyanidin-glucoside, Barley, Hordeum vulgare, Cyanidin and cyaniding glycosides, Bean, Pheseolus vulgaris (several cultivars), Cyanidin 3-glucoside, Cyanidin 3-glucoside, Cyanidin 3,5-diglucoside, Begonia semperflorens cvs, Cyanidin derivative, Benibana-cha, Camellia sinensis, Cyanidin 3-O-beta-D galactoside, Cyanidin 3-O-beta-D-galactoside, Bellis perennis, 3 Cyanidin 3-derivatives, Bletilla striata, Acylated cyanidin 3,7,3′-triglucoside derivatives, Bilberry, Vaccinium myrtillus, Artemis/Iprona; Indena, Cyanidin-3-galactoside (22%); Cyanidin-3-galactoside, Cyanidin-3-glucoside (9%), Cyanidin-3glucoside, Black beans, Phaseolus, Cyanidin-3-glucoside (96%), Cyanidin-3glucoside, Blackberry (European and American), Moriferi veri, Rubus caesius, R. Alleghniensis, R. argufus, R. cuneifolius, R. setosus, R. trivials, Cyanidin-glucoside (70-100%), Cyanidin-glucoside, Cyanidin-rutinoside, Black grapes, Many varieties, Black potatoes, Solanumtuberosum tuberosum, Cyanidin-glycosides, Black raspberry, Rubus occidentalis, Cyanidin-sambubloside (20%); Cyanidin-sambubloside, Cyanidin-xylosylrutinoside (40%); Cyanidin-glucoside, Cyanidin-glucoside, (17%), Cyanidin-rutinoside (23%), Black soybeans, Glycine max, Cyanidin-3-glucoside (96%), Cyanidin-3-glucoside, Blueberries, Five common Vaccinium spp, Cyanidin-glucoside (3%); Cyanidin-glucoside, Cyanidin-galactoside (3%), Cyanidin galactoside, Cyanidin-arabinoside (3%), Cyanidin-3-arabinoside, Bog whortleberry, Vaccinium uliginosum, Cyanidin-3-glucoside (14%), Cyanidin 3 glucoside (14%), Cyanidin #arabinoside (10%), Cyanidin-3-arabinoside (10%), Cyanidin 3-galactoside (6.5%), Cyanidin-3-galactoside (6.5%), Boysenberry, new Zealand, Cyanidin-3-sophoroside (44.5%), Cyanidin-3-glucoside, Cyanidin-3-glucoside (26.4%), Cyanidin-3 glycosylrutinoside (25.8%), Cyanidin-rutinoside (3.3%), Buckwheat, Fagopyrum species, Cyanidin-3-glucoside, Cyanidin-3-glucoside, Cyanidin 3-galactoside, Cyanidin-3-galactoside, Cacao, Theobroma cacao, Cyanidin 3-glucoside (suspected), Cyanidin-3-glucoside (suspected), Celery, Apium spp, Cherry laurel, Prunus laurocerasus, Cyanidin-3-arabinoside, Cyanidin-3-arabinoside, Chicory, Cichorium inrybus, Cyanidin 3-glucoside, Cyanidin 3-glucoside, Chive, Allium schoenoprasum, Cyanidin-3-glucoside, Cyanidin-3-glucoside, Cyanidin-3-acetylglucoside, Cyanidin 3-(6 malonylglucoside), Cyanidin 3-(3,6 dimalonylglucoside), Chokeberry, Aronia melanocarpa, Artemis/Iprona, Cyanidin-3-galactoside (64.5%), Cyanidin-3-galactoside, Cyanidin-3-arabinoside (28.9%), Cyanidin-3 arabinoside, Cyanidin-3-glucoside (2.4%), Cyanidin-3 glucoside, Cyanidin-3-xyloside (4.2%), Cyanidin-3-xyloside, Coffee, Cofea arabica cv. Bourbon Vermelho, Cyanadin-3-glycoside, Cyanadin 3,5-diglyeoside, Cyanadin 3-glycoside, Cotoneaster, Cotoneaster Medic. Spp, Cyanidin 3-glucoside, Cyanidin 3-glucoside, Cyanidin 3-galactoside, Cyanidin 3-rutinoside, Cyanidin 3 galactoside, Cowberry or Lingonberry, V. vitis-idaea, Cyanidin 3-galactoside Cyanidin 3-arabinoside, Cyanidin 3-galactoside, Cyanidin 3-glucoside, Cyanidin 3 arabinoside, Cyanidin 3 glucoside, Chimonanthus praecox, Cyanidin 3-O-glucoside, Cyanidin-3-O-glucoside, Acylated cyanidin 3-0-glucoside, Cyanidin glycoside, Cranberry (American and European), Vaccinium macrocorpon, Ocean Spray, Cyanidin-galactoside (16-24%), Cyanidin-galactoside, V. oxycoccus, Cyanidin-arabinoside (13-25%), Cyanidin arabinoside, CrOwberry, Empetrum nigrum, Cyanidin 3-glucoside Cyanidin 3,5-diglucoside, Cyanidin 3-glucoside, Cyanidin 3-rutinoside, Cyanidin 3-sophoroside, Chrysanthemum, Dendranthema Grandiflorum, Cyanidin 3-O-(6′-O-malonyl-beta-glucopyranoside, Currant (red and black), Ribes rubrum, R. nigrum, Cyanidin-glucoside (2-10%), Cyanidin-glucoside, Cyanidin sambubioside, Cyanidin-rutinoside (8-17%), Cyanidin-sambubioside (9-31%), Cyanidin-sophoroside (4-9%), Cyanidin xylosylrutinoside (28-73%), Cyanidin glucosylrutinoside (14-28%), Cyneinonurn coccineum, Cyanidin 3-O-glucoside (92%), Cyanidin 3-O-glucoside (92%), Cyanadin 3-O-(6-0 rhamnosylglucoside (8%), Danewort, Sambucus ebulus, Cyanidin 3-xylosylglucoside, Cyanidin 3-sambubioside, Cyanidin 3 sambubloside, Cyanidin 3-glucoside, Cyanidin 3-sambubioside-5-glucoside, Cyanidin 3,5-diglucoside, Cyanidin 3-glucoside, Cyanidin 3-arabinoglucoside, Dendrobium, Phalaenapsis spp, Cyanidin derivatives, Dwarf dogwood, Comus suecica, Cyanidin 3-glucoside (4%), Cyannidin 3-glucoside (4%), Cyanidin 3-galactoside (16%), 2 Cyanidin derivatives (80%), Echinacea, Echinacea spp., Eldenberry, Sambucus nigra, Artemis/Iprona, Cyanidin-3-glucoside (42%), Cyanidin-3-glucoside, Cyanidin-3-sambubioside (43%) Cyanidin-3,5-diglucoside (2%), Cyanidin-3 sambubloside-5 glucoside (9%), Gentians spp, Cyanidin 3-O-beta-D-glucoside and 3 other derivatives, Cyanidin 3-O-beta-D-glucoside, Fatsia japonica, Cyanidin 3-lathyroside, Feijoa, Feijoa sellowiana, Cyanidin 3-glucoside, Cyanidin 3-glucoside, Fig, Ficus carica spp, Cyanidin 3-rhamnoglucoside, Cyanidin 3,5-diglucoside, Cyanidin 3-glucoside, Forsythia X, intermedia cv, Spring Glory, Cyanidin derivatives, Garlic, Allium sativum, Cyanidin 3-glucoside, Cyanidin 3-glucoside, Cyanidin 3-glucoside, Cyanidin 3-glucoside monoacylated, Cyanidin 3-glucoside triacylated, Ginseng, Panax ginseng, Panax quinquefolius, Cyanidin 3-O-β-D-xylopyranyl-(12)-β-D-glucopyranoside, Globe artichoke, Cynara scolymus, Cyanidin 3-caffeylglucoside, Cyanidin 3-caffeylsophoroside, Cyanidin 3-dicaffeylsophoroside, Gooseberry, Ribes spp, Cyanidin 3-glucoside, Cyanidin 3-glucoside, Cyanidin 3-rutinoside, Grape, Vinis vinifera, Cyanidin 3-monoglucoside, Cyanidin 3-monoglucoside, Cyanidin 3-monoglucoside-acetate, Cyanidin 3-monoglucoside-p-coumarate, Guava, Psidium guajavica, Cyanidin 3-glucoside, Cyanidin 3-glucoside, Hawthorn, Crataegus spp, Cyanidin 3-galactoside, Cyanidin 3-galactoside, Cyanidin 3-arabinoside, Cyanidin 3-glucoside, Cyanidin 3 glucoside, Hibiscus or Roselle, Hibiscus sabdariffa, Cyanidin-sambubioside (30%), Hollyhock, Althaea rosea, Cyanidin 3-glucoside, Cyanidin 3-rutinoside, Cyanidin 3-glucoside, Other cyaniding glucosides, Honeysuckle, Lonicera nitida, Cyanidin 3-rutinoside, Cyanidin 3-glucoside, Cyanidin 3-glucoside, Japanese garden iris, Iris ensata, Cyanidin 3RG, Cyanidin 3RG5G, Cyanidin 3Rgac5G, Ipornoea purpurea, Six acylated cyanidin 3-sophoroside-5 glucosides, Java plum, Mytciana jaboticaba, Cyanidin 3-glucoside, Cyanidin 3-glucoside, Jerusalem artichoke, Helianthus tuberosus, Kokum, Garcinia indica, Cyanidin 3-glucoside, Cyanidin 3-glucoside, Cyanidin 3-sambubioside, Cyanidin 3-sambubioside, Laeliocattleya cv Mini purple, Acylated cyaniding derivatives, Lactuca saliva, Cyanidin 3-O-(6″-malonylglucoside), Loganberry, Rubus loganbaccus, Cyanidin-sophoroside (48.1%), Cyanidin-glucoside, Cyanidin-glucoside (21.6%), Cyanidin-rutinoside (6.2%), Lupine, Lupinus spp, Cyanidin glycosides, presence confirmed, Lychee, Litchi chinensis, Cyanidin 3-glucoside, Cyanidin 3-glucoside, Cyanidin 3-galactoside, Cyanidin 3-rutinoside, Cyanidin 3 galactoside, Maize, Zea mays, Cyanidin 3-glucoside, Cyanidin 3-glucoside, Cyanidin 3-(6″-malonylglucoside) Cyanidin 3(3″,6″dimalonyl-glucoside) Mango, Mangifera indica, (Cyanidin glycosides, Mangosteen, Garcina mangostana, Cyanidin 3-sophoroside, Cyanidin 3-glucoside, Cyanidin 3-glucoside, Maqul, Aristotella chilensis, Cyanidin 3-,5-diglucoside, Matthiola incana, Four acylated cyaniding 3-sambubloside-5 glucosides, Millet, Pernnisetum americanum, Cyanidin 3-glucoside, Cyanidin 3-glucoside, Mountain ash berry, Sorbus spp, Cyanidin 3-galactoside, Cyanidin 3,5-diglucoside Cyanidin 3-β-D glucopyranoside, Mulberry, Morus nigra, Cyanidin 3-glucoside, Cyanidin 3-glucoside, Cyanidin 3,5-diglucoside, Cyanidin 3-rutinoside, Cyanidin 3-sophoroside, Myrtle berry, Myrtus communis, Cyanidin 3-glucosides, Cyanidin 3-glucosides, Cyanidin 3-diglucosides, Olive, Olea europaea, Cyanidin 3-rutinoside, Cyanidin 3-glucoside, Cyanidin 3-glucoside, Cyanidin derivatives, Onion, Allium sepa, Cyanidin 3-glucoside, Cyanidin 3-glucoside, Cyanidin 3-diglucoside, Cyanidin 3-laminarioside, Orange, Citrus sinensis, Cyanidin 3-glucoside (95%), Cyanidin 3-glucoside, Cyanidin 3,5-diglucoside, Passion fruit, Pasiflora edulis, Cyanidin 3-glucoside, Cyanidin 3-glucoside, Pea, Pisum sativurn, Cyanidin 3-sophoroside glucosides, Cyanidin 3-sambubioside-5-glucosides, Peach, Prunus persica, Cyanidin 3-glucoside, Cyanidin 3-glucoside, Cyanidin 3-rutinoside, Cyanidin derivatives, Peanut, Arachis hypogaea, Cyanidin glucosides, Pear, Pyrus communis, Cyanidin 3-galactoside, Cyanidin 3-galactoside, Cyanidin 3-arabinoside, Cyanidin 3-arabinoside, Perilla, Perilla frutescens, Cyanidin 3,5-diglucoside, Cyanidin 3,5-derivatives, Petunia spp, Cyanidin 3-rutinoside, Phalsa, Grewia spp, Cyanidin 3-glucoside, Cyanidin 3-glucoside, Pineapple, Anans comosus, Cyanidin 3-galactoside, Cyanidin 3-galactoside, Pistachio, Pistacia vera, Pragmites australis, Cyanidin-3 derivatives, Plum, 2000 varieties, 15 species, Cyanidin-glucoside (37%), Cyanidin glucoside, Cyanidin-rutinoside (45%), Pomegranate, Punica granatam, Cyanidin-glucoside (30%), Cyanidin-glucoside, Cyanidin-diglucoside (17%), Purple carrot, Daucus carota, Cyanidin-glucoside, Cyanidin-glucoside, Cyanidin-glucosylgalactoside, Cyanidin-galactoside, Cyanidin-digalactoside, Cyanidin-galactoside, Quince, Cyclonia oblonga, Cyanidin-3 glucoside, Cyanidin 3,5-diglucoside, Cyanidin derivatives, Radish, Raphanus sativus, Acylated cyanidin 3-sophoroside-5-glucoside, Acylated cyanidin 3 diglucoside-5-glucoside, Red cabbage, Brassica oleracea var capitata, Cyanidin glycosides, Reed, Phalaris arundinacea, Cyanidin 3-glucoside, Cyanidin 3-glucoside, Cyanidin 3-(6″-,malonylglucoside), Cyanidin 3 (3″,6″dimalonyl-glucoside), Red onion, Allium cepa, Cyanidin 3-glucoside, Cyanidin 3-glucoside, Acylated cyanidin 3-glucoside derivatives, Red petunia, Petunia spp, Cyanidin 3-glucoside, Cyanidin 3-glucoside, Cyanidin 3-sophoroside, Red raspberry, Rubus idaeus, Cyanidin glucoside (17%), Cyanidin-glucoside, Cyanidin-rutinoside (7%), Cyanidin-sophoroside (50%), Cyanidin glycosylrutinoside (26%), Cyanidin-diglucoside, Rhubarb, Rneum spp, Cyanidin 3-glucoside, Cyanidin 3-glucoside, Cyanidin 3-rutinoside, Rice, Oryza spp, Cyanidin 3-glucoside, Cyanidin 3-glucoside, Cyanidin 3-rhamnoside, Cyanidin 3,5-diglucoside, Rosehip, Rosa canina, Cyanidin 3-rutinoside, Cyanidin 3-glucoside, Cyanidin 3-glucoside, Cyanidin 3,5-diglucoside, Rye, Secale cereale, Cyanidin 3-glucoside, Cyanidin 3-glucoside, Cyanidin 3-rhamnosylglucoside, Cyanidin 3-rhamnosyldiglucoside, Cyanidin 3-rutinoside, Cyanidin 3-rutinoside derivatives, Cyanidin 3-gentiobioside, Sheepberry, Viburnum spp, Cyanidin 3-glucoside, Cyanidin 3-glucoside, Cyanidin 3-arabinosylsambubioside, Sorghum, Sorghum bicolor, Cyanidin, Cyanidin glycosides, Sparkleberry, Varboreum, Cyanidin 3-glucoside, Cyanidin 3-glucoside, Cyanidin 3-arabinoside, Cyanidin 3-galactoside, Strawberry, Fragaria ananassa, Cyanidin-glucoside(minor), Cyanidin-glucoside, Sunflower, Hellanthus annuus, Cyanidin 3-glucoside, Cyanidin 3-glucoside, Acylated cyanidin 3-glucoside, Cyanidin 3-xyloside, Cyanidin 3-xyloside, Acylated cyanidin 3-xyloside, Cyanidin 3-vanillyl sambubioside, Sweet cherry, Prunus avintn, Cyanidin-glucoside, Cyanidin-glucoside, Cyanidin-rutinoside; Cyanidin 3-suphoroside, Sweet potato, Ipornoea batatas Sophronitis coccinea, Cyanidin derivatives, Five acylated cyanidin 3,3′,7-triglucosides, Tamarillo or tomato tree, Cyphomandrea betacea, Cyanidin 3-rutinoside, Cyanidin 3-glucoside, Cyanidin 3-glucoside, Tamarind, Tamarindus indica, Cyanidin 3-glucoside, Cyanidin 3-glucoside, Taro, Colocasia esculenta, Cyanidin 3-glucoside, Cyanidin 3-glucoside, Cyanidin 3-rutinoside, Tart Cherry (balaton), Prunus cerasus cv. Balaton, Nutrilite, Cyanidin-3-rutinoside-hexose (75%), Cyanidin-3-rutinoside-pentose (3%), Cyanidin-3-rutinoside (18%), Tart cherry (montmorency), Prunus cerasus cv. Montmorency, Nutrilite, Cyanidin-3-sophoroside (80%), Cyanidin-3-glucoside (20%), Cyanidin-3-glucoside (20%), Tulip, Tulipa spp, Cyanidin 3-O-(6″-rhamnosylglucosides), Cyanidin 3-O-derivative, Turnip, brissica rapa, Cyanidin 3-glucoside, Cyanidin 3-glucoside, Cyanidin 3-diglucoside-5-glucoside, Water lily, Nymnphasa alba, Cyanidin 3-O-(6″-acetyl-beta-galactopyrosinase (23%), Cyanidin 3-0-galactoside (2%), Cyanidin 3-O-galactoside (2%), Weigela spp, Cyanidin 3-O-glucoside, Cyanidin 3-O-glucoside, Cyanidin 3-O-glucoside xylose, Wheat, Triticum spp, Cyanidin 3-glucoside, Cyanidin 3-glucoside, Acylated cyanidin glucoside, Cyanidin 3-rutinoside, Acylated cyanidin 3-rutinoside, Cyanidin 3-gentiobioside, Wild rice, Zizania aquatica, Cyanidin 3-glucoside, Cyanidin 3-glucoside, Cyanidin 3-rhamnoglucoside, and Yam, Dioscoracea spp, Cyanidin 3,5-diglucoside, Cyanidin 3-glucoside, Cyanidin 3-glucoside, Cyanidin 3-rhamnoglucoside, Cyanidin 3-gentiobioside, Acylated cyanidin glucosides.

The term “anthocyanin” as used herein is intended to refer not only to monomeric anthocyanins, but also refers to dimeric and polymeric (i.e. containing from 3 to 20 anthocyanidin monomer residues) forms of anthocyanins and to leucoanthocyanidins (also known as flavan-3,4-diols). The anthocyanins can comprise substitutions (e.g. alkyl, alkoxy groups etc.) and in particular can be O-glycosylated, as described above. The anthocyanin in the dispersible colorant can be a single anthocyanin, or comprise a mixture of anthocyanins. In particular, the anthocyanin is selected from the group consisting of: malvidin, cyanidin, delphinidin, paeonidin, pelargonidin and petunidin, and glycosides thereof A typical example is malvin (malvidin diglucoside) chloride, which is commercially available in a purified form. Alternatively the anthocyanin can be obtained by extracting anthocyanin containing plants such as grape, black carrot, red cabbage, blackberry, blackcurrent, cranberry and the like as described above.

In one embodiment, the colorant is selected from natural colorants. In an another embodiment, the colorant is selected from synthetic colorants. In still another embodiment, the colorant is selected from water-soluble colorants. In yet another embodiment, the colorant is selected from oil-soluble colorants. In still yet another embodiment, the colorant is selected from edible colorants. Water soluble edible colorants include, for example, safflower yellow, sweet potato red, Anthoblue, sodium copper chlorophyllin, gardenia blue, carminic acid, elderberry, red radish, beet root red, melanin, aronia and mixtures thereof. Suitable oil-soluble edible colorants include, for example, curcumin, paprika red, lutein, β-carotene, lycopene and mixtures thereof.

It is known in the art that any color can be obtained from the 3 primary colors, i.e. yellow, blue, and red. Therefore, in one aspect, a dispersible colorant comprising a combination of two or more colorants compounding with each other and at least one polysaccharide is provided. In another aspect, the two or more colorants can be compounded with each other before being combined with the polysaccharide. In still another aspect, the two or more colorants can be compounded with each other after being combined with the polysaccharide respectively.

As used herein, the term “polysaccharide” refers to long carbohydrate molecules of repeated monomer units joined together by glycosidic bonds. Specific examples of polysaccharides include, but not limited to, starches and starch derivatives, glycogen, celluloses and cellulose derivatives, chitin, and pectin.

As used herein, the term “starch” and “starch derivatives” refers to starch and starch derivatives from any source, including, but not limited to, synthetic sources and natural sources.

Starch from natural sources include, but are not limited to, corn starch, potato starch, rice starch, wheat starch, etc.

Starch derivatives include, but are not limited to carboxmethyl starch, hydropropyl starch, crosslinked derivatives thereof, salts thereof, etc.

In one aspect, the polysaccharide is selected from the group consisting of celluloses and their derivatives. In another aspect, the polysaccharide is selected from edible celluloses or starches that are crosslinked and their derivatives.

As used herein, the term “cellulose” and “cellulose derivatives” refers to celluloses and cellulose derivatives from any sources, including, but not limited to, synthetic sources and natural sources.

Celluloses from natural sources include, but are not limited to those from, vegetables, such as celery, potato, etc.; fruits, such as apple, banana, etc.; bamboo; cotton; and bast, such as linum, cannabis, nettle, boehmeria, etc.

Celluloses from synthetic sources include modified cellulose derivatives, including, but not limited to: cellulose esters, such as cellulose acetate, cellulose triacetate, cellulose propionate, cellulose acetate propionate, cellulose acetate butyrate, cellulose nitrate, cellulose sulfate, etc.; cellulose ethers, such as methylcellulose, ethylcellulose, ethyl methyl cellulose, hydroxyethyl cellulose, hydropropyl cellulose, hydroxyethyl methyl cellulose, hydroxypropyl methyl cellulose (HPMC), crosslinked hydroxypropyl methyl cellulose, ethyl hydroxyethyl cellulose, carboxymethylcellulose (CMC), crosslinked carboxymethyl cellulose, sodium carboxymethylcellulose, crosslinked sodium carboxymethylcellulose, etc.; and microcrystalline cellulose, etc. In particular embodiments, the cellulose is selected from crosslinked cellulose derivatives.

It should be understood that throughout the specification the term “solution” is used. Solution, as used herein, should not be construed as limiting and includes mixtures, emulsions, suspensions, homogenous systems and heterogeneous systems, etc.

The term “precipitate” as used herein is intended to include solid materials that are isolated from the processes described herein. Precipitates include, for example, complexes, colloids, solids, etc.

In one aspect, a dispersible colorant comprising a combination of at least one colorant and at least one polysaccharide is provided, in which the colorants are adsorbed on the polysaccharide or are combined with the polysaccharide.

Accordingly, the colorants can be adsorbed on the polysaccharides by any process known in the art. Such methods include, but are not limited to, physical adsorption, chemical adsorption, interpenetration, complexation, electrostatic interations, and/or combinations thereof.

Therefore, a process is provided for preparing the dispersible colorants described herein comprising a combination of at least one colorant and at least one polysaccharide, wherein the colorants are adsorbed and/or are intimately dispersed on and or within the polysaccharides.

The dispersible colorants described herein can be provided in various forms and incorporated into various products such as in food stuffs, nutraceuticals and with a drug.

The dispersible colorants can be incorporated into various foods, drinks, snacks, etc. In one aspect, the dispersible colorant can be sprinkled onto a food product, prior to consumption. If sprinkled onto a food product, a suitable carrier such as starch, sucrose or lactose, can be used to help distribute the concentration of the dispersible colorants making it easier to apply to the food product.

The dispersible colorants described herein can also be provided in supplements as the prepared food products. For the purposes of this application, prepared food product means any natural, processed, diet or non-diet food product to which a dispersible colorant has been added. The dispersible colorant can be directly incorporated into many prepared diet food products, including, but not limited to beverages, such as milk, water, diet drinks, sodas, beer, alcoholic beverages, such as vodka, gin, etc., diet bars and prepared frozen meals. Furthermore, the dispersible colorants described herein can be incorporated into many prepared non-diet products, including, but not limited to candy, snack products such as chips, prepared meat products, milk, cheese, yogurt, sport bars, sport drinks, mayonnaise, salad dressing, bread and any other fat or oil containing foods. As used herein, the term “food product” refers to any substance fit for human or animal consumption.

The dispersible colorants described herein can be added to various drinks, such as fruit juices, milkshakes, milk, etc.

One method of administration is oral. The dispersible colorants can be formulated with suitable carriers such as starch, sucrose or lactose in tablets, capsules, granuales, injectibles (intravenous, etc.) solutions, syrups and emulsions. The tablet or capsule of can be coated with an enteric coating that dissolves at a pH of about 6.0 to 7.0. A suitable enteric coating, which dissolves in the small intestine but not in the stomach, is cellulose acetate phthalate.

Formulation of the dispersible colorants described herein can be placed into a soft gel capsule can be accomplished by many methods known in the art. Often the formulation will include an acceptable carrier, such as an oil, or other suspending or emulsifying agent.

Suitable optional carriers include but are not limited to, for example, fatty acids, esters and salts thereof, that can be derived from any source, including, without limitation, natural or synthetic oils, fats, waxes or combinations thereof. Moreover, the fatty acids can be derived, without limitation, from non-hydrogenated oils, partially hydrogenated oils, fully hydrogenated oils or combinations thereof. Non-limiting exemplary sources of fatty acids (their esters and salts) include seed oil, fish or marine oil, canola oil, vegetable oil, safflower oil, sunflower oil, nasturtium seed oil, mustard seed oil, olive oil, sesame oil, soybean oil, corn oil, peanut oil, cottonseed oil, rice bran oil, babassu nut oil, palm oil, low erucic rapeseed oil, palm kernel oil, lupin oil, coconut oil, flaxseed oil, evening primrose oil, jojoba, wheat germ oil, tallow, beef tallow, butter, chicken fat, lard, dairy butterfat, shea butter or combinations thereof.

Specific non-limiting exemplary fish or marine oil sources include shellfish oil, tuna oil, mackerel oil, salmon oil, menhaden, anchovy, herring, trout, sardines or combinations thereof. In particular, the source of the fatty acids is fish or marine oil (DHA or EPA), soybean oil or flaxseed oil. Alternatively or in combination with one of the above identified carrier, beeswax can be used as a suitable carrier, as well as suspending agents such as silica (silicon dioxide).

The dispersible colorants described herein can also be included in a nutraceutical. The term “nutraceutical” is recognized in the art and is intended to describe specific chemical compounds found in foods that can prevent disease or ameliorate an undesirable condition.

The dispersible colorants described herein can further include various ingredients to help stabilize, or help promote the bioavailability of the components of the beneficial aspects of the composition or serve to help provide additional nutrients to an individual's diet. Suitable additives can include vitamins and biologically-acceptable minerals. Non-limiting examples of vitamins include vitamin A, B vitamins, vitamin C, vitamin D, vitamin E, vitamin K and folic acid. Non-limiting examples of minerals include iron, calcium, magnesium, potassium, copper, chromium, zinc, molybdenum, iodine, boron, selenium, manganese, derivatives thereof or combinations thereof. These vitamins and minerals can be from any source or combination of sources, without limitation. Non-limiting exemplary B vitamins include, without limitation, thiamine, niacinamide, pyridoxine, riboflavin, cyanocobalamin, biotin, pantothenic acid or combinations thereof.

Various additives can be incorporated into the compositions described herein with the dispersible colorant(s). Optional additives of the present compositions include, without limitation, hyaluronic acid, phospholipids, starches, sugars, fats, antioxidants, amino acids, proteins, flavorings, coloring agents, hydrolyzed starch(es) and derivatives thereof or combinations thereof.

As used herein, the term “antioxidant” is recognized in the art and refers to synthetic or natural substances that prevent or delay the oxidative deterioration of a compound. Exemplary antioxidants include tocopherols, flavonoids, catechins, superoxide dismutase, lecithin, gamma oryzanol; vitamins, such as vitamins A, C (ascorbic acid) and E and beta-carotene, natural components such as camosol, camosic acid and rosmanol found in rosemary and hawthorn extract, proanthocyanidins such as those found in grapeseed or pine bark extract, and green tea extract.

Compositions comprising the dispersible colorants described herein can be manufactured by methods of conventional mixing, dissolving, granulating, dragee-making levigating, emulsifying, encapsulating, entrapping or lyophilization processes. The compositions can be formulated in conventional manner using one or more physiologically acceptable carriers, diluents, excipients or auxiliaries that facilitate processing of the compositions into preparations that can be used.

The compositions that include the dispersible colorants described herein can take a form suitable for virtually any mode of administration, including, for example, oral, buccal, systemic, injection, transdermal, rectal, vaginal, etc., or a form suitable for administration by inhalation or insufflation.

Systemic formulations include those designed for administration by injection, e.g., subcutaneous, intravenous, intramuscular, intrathecal or intraperitoneal injection, as well as those designed for transdermal, transmucosal oral or pulmonary administration.

Useful injectable preparations include sterile suspensions, solutions or emulsions of the compositions in aqueous or oily vehicles. The compositions can also contain formulating agents, such as suspending, stabilizing and/or dispersing agent. The formulations for injection can be presented in unit dosage form, e.g., in ampoules or in multidose containers, and can contain added preservatives.

Alternatively, the injectable formulation can be provided in powder form for reconstitution with a suitable vehicle, including but not limited to sterile pyrogen free water, buffer, dextrose solution, etc., before use. To this end, the compositions can be dried by any art-known technique, such as lyophilization, and reconstituted prior to use.

For transmucosal administration, penetrants appropriate to the barrier to be permeated are used in the formulation. Such penetrants are known in the art.

For oral administration, the compositions described herein that contain a dispersible colorant can take the form of, for example, lozenges, tablets or capsules prepared by conventional means with pharmaceutically acceptable excipients such as binding agents (e.g., pregelatinised maize starch, polyvinylpyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycolate); or wetting agents (e.g., sodium lauryl sulfate). The tablets can be coated by methods well known in the art with, for example, sugars, films or enteric coatings.

Liquid preparations for oral administration can take the form of, for example, elixirs, solutions, syrups or suspensions, or they can be presented as a dry product for constitution with water or other suitable vehicle before use. Such liquid preparations can be prepared by conventional means with pharmaceutically acceptable additives such as suspending agents (e.g., sorbitol syrup, cellulose derivatives or hydrogenated edible fats); emulsifying agents (e.g., lecithin or acacia); non aqueous vehicles (e.g., almond oil, oily esters, ethyl alcohol, or fractionated vegetable oils); and preservatives (e.g., methyl or propyl p hydroxybenzoates or sorbic acid). The preparations can also contain buffer salts, preservatives, flavoring, coloring and sweetening agents as appropriate.

Preparations for oral administration can be suitably formulated to give controlled release of the composition as is well known.

For buccal administration, the compositions can take the form of tablets or lozenges formulated in conventional manner.

For rectal and vaginal routes of administration, the compositions can be formulated as solutions (for retention enemas) suppositories or ointments containing conventional suppository bases such as cocoa butter or other glycerides.

For nasal administration or administration by inhalation or insufllation, the compositions can be conveniently delivered in the form of an aerosol spray from pressurized packs or a nebulizer with the use of a suitable propellant, e.g., dichlorodifluoromethane, trichlorofluoromethane, dichlorotetrafluoroethane, fluorocarbons, carbon dioxide or other suitable gas. In the case of a pressurized aerosol, the dosage unit can be determined by providing a valve to deliver a metered amount. Capsules and cartridges for use in an inhaler or insufflators (for example capsules and cartridges comprised of gelatin) can be formulated containing a powder mix of the compound and a suitable powder base such as lactose or starch.

For prolonged delivery, the compositions can be formulated as a depot preparation for administration by implantation or intramuscular injection. The compositions can be formulated with suitable polymeric or hydrophobic materials (e.g., as an emulsion in an acceptable oil) or ion exchange resins, or as sparingly soluble derivatives, e.g., as a sparingly soluble salt. Alternatively, transdermal delivery systems manufactured as an adhesive disc or patch, which slowly releases the compositions for percutaneous absorption, can be used. To this end, permeation enhancers can be used to facilitate transdermal penetration of the compositions. Suitable transdermal patches are described in for example, U.S. Pat. No. 5,407,713; U.S. Pat. No. 5,352,456; U.S. Pat. No. 5,332,213; U.S. Pat. No. 5,336,168; U.S. Pat. No. 5,290,561; U.S. Pat. No. 5,254,346; U.S. Pat. No. 5,164,189; U.S. Pat. No. 5,163,899; U.S. Pat. No. 5,088,977; U.S. Pat. No. 5,087,240; U.S. Pat. No. 5,008,110; and U.S. Pat. No. 4,921,475.

Alternatively, other delivery systems can be employed. Liposomes and emulsions are well-known examples of delivery vehicles that can be used to deliver compositions described throughout Certain organic solvents such as dimethylsulfoxide (DMSO) can also be employed, although usually at the cost of greater toxicity.

The compositions that contain the dispersible colorant can, if desired, be presented in a pack or dispenser device, which can contain one or more unit dosage forms containing the dispersible colorant and an active ingredient. The pack can, for example, comprise metal or plastic foil, such as a blister pack. The pack or dispenser device can be accompanied by instructions for administration.

In one embodiment, the process for preparing the combination of at least one colorant and at least one polysaccharide comprises the steps of:

1) weighing a predetermined amount of a water-soluble colorant, and dissolving it in water, in order to form a solution with a predetermined concentration, wherein the predetermined amount is in the range of greater than zero to a point of saturation of the colorant in the solvent, under the conditions of the dissolving process;

2) adding an appropriate amount of a polysaccharide into the solution prepared in step 1), wherein the amount of the polysaccharide is determined by a requirement, such as chromaticity, of the desired final product;

3) optionally adding a pH regulator until the pH reaches 2-8.5, allowing the components to interact for 1 min to 10 h, such as 1 min, 2 min, 3 min, 5 min, 10 min, 15 min, 20 min, 30 min, 1 h, 2 h, 3 h, 5 h or 10 h, until the carrier, such as the polysaccharide, is completely dissolved;

4) heating the obtained solution, mixture or suspension from step 3, for 1 min to 10 h, such as 1 min, 2 min, 3 min, 5 min, 10 min, 15 min, 20 min, 30 min, 1 h, 2 h, 3 h, 5 h or 10 h, in a water-bath at a temperature of room temperature to 100° C., such as 25° C., 30° C., 35° C., 40° C., 50° C., 60° C., 70° C., 80° C., 90° C. and 100° C. to afford a colored precipitate;

5) separating the colored precipitate, (generally after cooling to room temperature or below) and washing several times with water;

6) drying the obtained precipitate; and

7) optionally grinding and sieving the precipitate, to provide a final composition.

In an another embodiment, the process for preparing the combination of at least one colorant and at least one polysaccharide comprises the steps of:

1) weighing a predetermined amount of an oil-soluble colorant, and dissolving it in an organic solvent or mixture of organic solvents, in order to form a solution with a predetermined concentration, wherein the predetermined amount is in the range of greater than zero to a point of saturation of the colorant in the solvent or mixture of organic solvents under the conditions of the dissolving process;

2) adding an appropriate amount of water and surfactant while stirring extensively until a homogenous solution is formed;

3) adding an appropriate amount of a polysaccharide to the homogenous solution, allowing the components to interact for 1 min to 10 h, such as 1 min, 2 min, 3 min, 5 min, 10 min, 15 min, 20 min, 30 min, 1 h, 2 h, 3 h, 5 h or 10 h;

4) optionally adding a pH regulator until the pH reaches 2-8.5, from step 3) and allowing the components to interact for 1 min to 10 h, such as 1 min, 2 min, 3 min, 5 min, 10 min, 15 min, 20 min, 30 min, 1 h, 2 h, 3 h, 5 h or 10 h, until the polysaccharide is dissolved;

(5) heating the solution of step 4) to a temperature of 30-100° C., such as 30° C., 35° C., 40° C., 45° C., 50° C., 60° C., 70° C., 80° C., 90° C., and 100° C., while stirring for 1 min to 10 h, such as 1 min. 2 min, 3 min. 5 min, 10 min, 15 min, 20 min, 30 min, 1 h, 2 h, 3 h, 5 h or 10 h to form a colored precipitate;

(6) separating the colored precipitate, (generally after cooling to room temperature or below) and washing several times with water;

(7) drying the colored precipitate, and

(8) optionally grinding and sieving the dried precipitate to provide a composition.

In another aspect, a dispersible colorant comprising the combination of at least one colorant and at least one polysaccharide is provided, in which the colorants are encapsulated or coated by the polysaccharides.

Accordingly, the colorants can be encapsulated or coated by the polysaccharides by any process known in the art, including, but not limit to, acid precipitation, metal ion precipitation, adsorption, covalent binding, crosslinking, film-forming and/or combinations thereof.

Therefore, in another aspect, a process for preparing the combination of at least one colorant and at least one polysaccharide is provided, wherein the colorants are encapsulated or coated by the polysaccharides.

In another embodiment, a process for preparing the combinations of at least one colorant and at least one polysaccharide is provided that comprises the steps of:

1) weighing a predetermined amount of a polysaccharide, and dissolving it in water until the polysaccharide is dissolved to provide a solution;

2) adding a colorant to the solution until the colorant is dispersed in the solution to provide a colored solution;

3) optionally adding an appropriate amount of a pH regulator to the colored solution, until the pH regulator is dissolved, thus forming a colored precipitate;

4) drying the colored precipitate; and

5) optionally grinding and sieving the colored precipitate, to provide a composition.

In still another embodiment, a process for preparing the combination of at least one colorant and at least one polysaccharide is provided that comprises the steps of:

1) preparing an aqueous solution of a colorant and an aqueous solution of a metal salt, respectively;

2) adding a polysaccharide to the colorant solution prepared in step 1) at a temperature of 0-70° C., such as room temperature, 0° C., 10° C., 20° C., 30° C., 40° C., 50° C., 60° C., or 70° C., allowing the components to interact for 1 min to 10 h, such as 1 min, 2 min, 3 min, 5 min, 10 min, 15 min. 20 min, 30 min, 1 h, 2 h, 3 h, 5 h or 10 h to provide a mixture;

3) adding the aqueous solution of metal salt prepared in step 1) into the mixture obtained in step 2), such that a colored precipitate is formed from the mixture;

4) continuing to add the aqueous solution of metal salt to the mixture while monitoring the viscosity of the mixture, until the viscosity of the mixture is similar or equal to water;

5) washing the colored precipitate with water,

6) drying the colored precipitate, and

7) optionally grinding and sieving the precipitate.

As used herein, the phrase “allowing the components to interact” includes that the components can simply interact without further physical manipulation, or stirring, shaking, vortexing and the like. It is not meant to be limiting, only that the components are combined and can react with each other.

As used herein, the term “pH regulator” refers to an agent added to change or maintain pH, and can be selected from the group consisting of organic acids, mineral acids, organic bases, mineral bases, naturalizing agents, and buffering agents. Specific examples of pH regulators include, but not limited to, tartaric acid, oxalic acid, citric acid, and malic acid. In one aspect, the pH regulator is selected from an edible pH regulator, such as citric acid, lactic acid, tartaric acid, malic acid and salts thereof.

The separation of the precipitate can be carried out by any process known in the art, including, but not limited to, flocculation, filtration, centrifugation, crystallization, etc. In one aspect, the separation is carried out by filtration. In another aspect, the separation is carried out by centrifugation.

The drying process can be carried out by any process known in the art, including, but not limited to freeze-drying, air-drying, spray-drying, etc. The drying process can be carried out under either atmospheric pressure or at a reduced pressure. The drying process can be carried out under a temperature of −50° C. to 100° C., such as −50° C., −25° C., 0° C., 20° C., 30° C., 35° C., 40° C., 45° C., 50° C., 55° C., 60° C., 70° C., 80° C., and 100° C.

Organic solvents suitable for use include, but are not limited to, alcohols, such as methanol, ethanol, propanol, etc; ethers, such as ethyl ether, isopropyl ether, etc.; esters, such as ethyl acetate, butyl acetate, etc. In one aspect, the organic solvent is selected from edible organic solvents, such as ethanol, ethyl acetate, etc.

Surfactants that are suitable for use as described herein include, but are not limited to, anionic surfactants, such as alkyl sulfates, phosphates, carboxylates, etc.; cationic surfactants, such as cetyl trimethylammonium bromide, cetylpyridinium chloride, etc.; zwitterionic surfactants, such as cocamidopropyl betaine, lecithin, etc.; and nonionic surfactants, such as fatty alcohols, polyoxyethylene glycol alkyl ethers, etc. In one aspect, the surfactant is selected from edible surfactants, such as glycerides, phospholipids, dextrins, fatty acid esters, chitosan, saponins, glycosides, gums collagen, etc.

Metal salts suitable for use include, but are not limited to, salts of Ca²⁺, Cu⁺, Cu²⁺, Fe²⁺, Fe³⁺, Zn²⁺, Al³⁺, Mg²⁺, etc.

In the processes described herein, the precipitate can be optionally ground and/or sieved by a sieve of 100-300 mesh, such as 150-250 mesh, 100 mesh, 150 mesh, 180 mesh, 190 mesh, 200 mesh, 210 mesh, 220 mesh, 230 mesh, 240 mesh, 250 mesh, 300 mesh, etc., in order to reduce the particle size of the final product to a desired size.

According to the description provided herein, the polysaccharide can be dyed by the colorant by any process known in the art.

In the specification and the claims, the terms “including” and “comprising” are open-ended terms and should be interpreted to mean “including, but not limited to . . . .” These terms encompass the more restrictive terms “consisting essentially of” and “consisting of.”

It must be noted that as used herein and in the appended claims, the singular forms “a”, “an”, and “the” include plural reference unless the context clearly dictates otherwise. As well, the terms “a” (or “an”), “one or more” and “at least one” can be used interchangeably herein. It is also to be noted that the terms “comprising”, “including”, “characterized by” and “having” can be used interchangeably.

Unless defined otherwise, all technical and scientific terms used herein have the same meanings as commonly understood by one of ordinary skill in the art to which this invention belongs. All publications and patents specifically mentioned herein are incorporated by reference in their entirety for all purposes including describing and disclosing the chemicals, instruments, statistical analyses and methodologies which are reported in the publications which might be used in connection with the invention. All references cited in this specification are to be taken as indicative of the level of skill in the art. Nothing herein is to be construed as an admission that the invention is not entitled to antedate such disclosure by virtue of prior invention.

The following paragraphs enumerated consecutively from 1 through 68 provide for various aspects of the subject matter described herein. In one embodiment, in a first paragraph (1), a dispersible colorant comprising a combination of a colorant and a polysaccharide is provided, wherein the weight ratio of total colorant to total polysaccharide is in the range of 5000:1 to 1:5000. The dispersible colorant can be considered a complex, a coated material, an encapsulated material, absorbed material, adsorbed material, or a gelatinized material. That is the colorant and the polysaccharide are intimately dispersed amongst each other such that a unique composition is formed that results as the ultimate colorant composition useful in lipophilic or aqueous environments.

2. The dispersible colorant according to paragraph 1, wherein the weight ratio of total colorant to total polysaccharide is in the range of 1:1000-1000:1.

3. The dispersible colorant according to paragraph 2, wherein the weight ratio of total colorant to total polysaccharide is in the range of 1:100-100:1.

4. The dispersible colorant according to paragraph 3, wherein the weight ratio of total colorant to total polysaccharide is in the range of 1:10-10:1.

5. The dispersible colorant according to paragraph 1, wherein the weight ratio of total colorant to total polysaccharide is in the range of 1:5000, 1:500, 1:200, 1:100, 1:50, 1:10, 1:1, 10:1, 50:1, 100:1, 200:1, 500:1, 1000:1, or 5000:1.

6. The dispersible colorant according to any one of paragraphs 1-5, wherein the colorant is selected from synthetic colorants.

7. The dispersible colorant according to paragraph 6, wherein the colorant is selected from water-soluble colorants.

8. The dispersible colorant according to paragraph 7, wherein the colorant is selected from amaranth, sunset yellow, erythrosine, tartrazine, new red, indigo, and brilliant blue.

9. The dispersible colorant according to paragraph 6, wherein the colorant is selected from oil-soluble colorants.

10. The dispersible colorant according to paragraph 9, wherein the colorant is sudan III.

11. The dispersible colorant according to any one of paragraphs 1-5, wherein the colorant is selected from natural colorants.

12. The dispersible colorant according to paragraph 11, wherein the colorant is selected from carotenoids, flavonoids, quinone colorants, porphyrin colorants, betacyanines, dione colorants, monascus colorants, caramel colorants, gardenia colorants, and phycocyanin colorants.

13. The dispersible colorant according to paragraph 11, wherein the colorant is selected from Red Chilli Color, Annatto, and Gardenia Yellow.

14. The dispersible colorant according to paragraph 11, wherein the colorant is selected from Beefsteak Plant Color, Corn Color, Safflower Yellow, Red Cabbage Color, Red Sweet Potato Color, Purple Carrot Color, Elderberry Color, Aronia Color, Red Raddish Color, Grape Skin Color, Bilberry Color, Blackcurrant Color.

15. The dispersible colorant according to paragraph 11, wherein the colorant is selected from cochineal colorants, carminic acid and Lac Dye.

16. The dispersible colorant according to paragraph 11, wherein the colorant is Chlorophyll.

17. The dispersible colorant according to paragraph 11, wherein the colorant is betalain.

18. The dispersible colorant according to paragraph 11, wherein the colorant is curcumin.

19. The dispersible colorant according to paragraph 11, wherein the colorant is monascorubrin.

20. The dispersible colorant according to paragraph 11, wherein the colorant is caramel.

21. The dispersible colorant according to paragraph 11, wherein the colorant is gardenia blue.

22. The dispersible colorant according to paragraph 11, wherein the colorant is Spirulina Blue Color.

23. The dispersible colorant according to paragraph 11, wherein the colorant is an extract, concentrate, or juice from a plant or an animal.

24. The dispersible colorant according to paragraph 23, wherein the colorant is an extract, concentrate, or juice from saffron, safflower, gardenia, bilberry, sweet potato, red cabbage, carrot, or grape.

25. The dispersible colorant according to any one of paragraphs 1-5, wherein the polysaccharide is selected from starches and their derivatives, glycogen, celluloses and their derivatives, chitin, and pectin.

26. The dispersible colorant according to paragraph 25, wherein the starch is selected from corn starch, potato starch, rice starch and wheat starch.

27. The dispersible colorant according to paragraph 25, wherein the starch derivative is selected from carboxmethyl starch, hydropropyl starch, crosslinked derivatives thereof, and salts thereof.

28. The dispersible colorant according to paragraph 25, wherein the cellulose is from vegetables, fruits, bamboo, cotton, and bast.

29. The dispersible colorant according to paragraph 28, wherein the vegetable is selected from celery and potato.

30. The dispersible colorant according to paragraph 28, wherein the fruit is selected from apple and banana.

31. The dispersible colorant according to paragraph 28, wherein the bast is selected from linum, cannabis, nettle, and boehmeria.

32. The dispersible colorant according to paragraph 25, wherein the cellulose derivative is selected from cellulose esters, cellulose ethers, and microcrystalline cellulose.

33. The dispersible colorant according to paragraph 32, wherein the cellulose ester is selected from cellulose acetate, cellulose triacetate, cellulose propionate, cellulose acetate propionate, cellulose acetate butyrate, cellulose nitrate, and cellulose sulfate.

34. The dispersible colorant according to paragraph 32, wherein the cellulose ether is selected from methylcellulose, ethylcellulose, ethyl methyl cellulose, hydroxyethyl cellulose, hydropropyl cellulose, hydroxyethyl methyl cellulose, hydroxypropyl methyl cellulose, crosslinked hydroxypropyl methyl cellulose, ethyl hydroxyethyl cellulose, carboxymethylcellulose, crosslinked carboxymethyl cellulose, sodium carboxymethylcellulose, and crosslinked sodium carboxymethylcellulose.

35. The dispersible colorant according to paragraph 25, wherein the cellulose derivative is selected from crosslinked cellulose derivative.

36. The dispersible colorant according to any one of paragraphs 1-35, wherein the colorant is adsorbed on the polysaccharide.

37. The dispersible colorant according to paragraph 36, wherein the combination of the colorant and the polysaccharide is prepared by:

1) dissolving a water-soluble colorant in water to form a solution, wherein the colorant is present from an amount greater than zero to saturation in the water,

2) adding a polysaccharide to the solution prepared in step 1) to form a mixture;

3) optionally adding a pH regulator to the mixture of step 2) until the pH of the mixture reaches a pH of about 2 to about 8.5 to form a mixture;

4) heating the mixture obtained from step 3), followed by cooling the mixture until a colored precipitate is formed; and

5) separating the colored precipitate from the mixture;

6) optionally, washing the colored precipitate several times with water; and/or

7) optionally, drying the colored precipitate; and/or

8) optionally, grinding and/or sieving, the colored precipitate.

38. The dispersible colorant according to paragraph 36, wherein the combination of the colorant and the polysaccharide is prepared by:

1) dissolving an oil-soluble colorant in an organic solvent or a mixture of organic solvents to form a mixture, wherein the colorant is present from an amount greater than zero to saturation in the organic solvent(s);

2) adding a surfactant to the mixture of step 1), optionally with water to form a mixture;

3) adding a polysaccharide to the mixture of step 2) to form a mixture;

4) optionally adding a pH regulator to the mixture of step 3) until the pH of the mixture reaches a pH of about 2 to about 8.5 to form a mixture;

5) heating the mixture of step 4) to a temperature, followed by cooling the mixture until a colored precipitate is formed;

6) separating the colored precipitate from the mixture;

7) optionally, washing the colored precipitate several times with water, and/or

8) optionally, drying the colored precipitate; and/or

9) optionally, grinding and/or sieving, the colored precipitate.

39. The dispersible colorant according to any one of paragraphs 1-35, wherein the colorant is encapsulated or coated by the polysaccharide.

40. The dispersible colorant according to paragraph 39, wherein the combination of the colorant and the polysaccharide is prepared by:

1) dissolving the polysaccharide in water until the polysaccharide is completely dissolved;

2) adding the colorant to the solution of step 1) until the colorant is uniformly dispersed in the solution;

3) optionally adding the pH regulator to the solution of step 2);

4) forming the colored precipitate from the solution;

5) optionally drying the colored precipitate; and

6) optionally grinding and/or sieving the colored precipitate.

41. The dispersible colorant according to paragraph 39, wherein the combination of the colorant and the polysaccharide is prepared by:

1) preparing the aqueous solution of colorant and an aqueous solution of a metal salt, respectively;

2) dissolving the polysaccharide in the colorant solution prepared in step 1);

3) adding the aqueous solution of metal salt prepared in step 1) to the mixture of step 2) such that a colored precipitate forms, wherein, for example, the aqueous metal solution is added until the viscosity of the solution is similar or equal to water;

4) isolating the colored precipitate;

5) optionally, washing the colored precipitate with water and/or,

6) drying the colored precipitate, and/or

7) optionally grinding and/or sieving the colored precipitate.

42. The dispersible colorant according to any one of paragraphs 1-35, wherein the polysaccharide is dyed by the colorant.

43. Use of the dispersible colorant according to any one of paragraphs 1-42 in applications wherein conventional colorants can be applied.

44. The use according to paragraph 43, wherein the dispersible colorant is applied in the manufacture of food products, sweets, cosmetics, toys, and pharmaceutical products.

45. The dispersible colorant according to any one of paragraphs 37-41, wherein the temperature of the reaction medium after the addition of the polysaccharide is maintained from about 65° C. to about 85° C. for from about 5 minutes to about 8 hours, particularly from about 20 minutes to about 40 minutes.

46. A method to prepare a dispersible colorant, comprising the steps:

1) dissolving a water-soluble colorant in water to form a solution, wherein the colorant is present from an amount greater than zero to saturation in the water;

2) adding a polysaccharide to the solution prepared in step 1) to form a mixture;

3) optionally adding a pH regulator to the mixture of step 2) until the pH of the mixture reaches a pH of about 2 to about 8.5 to form a mixture;

4) heating the mixture obtained from step 3), for example, for about 1 minute to about 10 hours at a temperature of from about room temperature to about 100° C., followed by cooling the mixture until a colored precipitate is formed; and

5) separating the colored precipitate from the mixture;

6) optionally, washing the colored precipitate several times with water, and/or

7) optionally, drying the colored precipitate; and/or

8) optionally, grinding and/or sieving, the colored precipitate.

47. A method to prepare a dispersible colorant, comprising the steps:

1) dissolving an oil-soluble colorant in an organic solvent or a mixture of organic solvents to form a mixture, wherein the colorant is present from an amount greater than zero to saturation in the organic solvent(s);

2) adding a surfactant to the mixture of step 1), optionally with water to form a mixture;

3) adding a polysaccharide to the mixture of step 2) to form a mixture;

4) optionally adding a pH regulator to the mixture of step 3) until the pH of the mixture reaches a pH of about 2 to about 8.5 to form a mixture;

(5) heating the mixture of step 4) to a temperature, for example, of about 30° C. to about 100° C. for about 1 minute to about 10 hours, followed by cooling the mixture until a colored precipitate is formed;

6) separating the colored precipitate from the mixture;

7) optionally, washing the colored precipitate several times with water; and/or

8) optionally, drying the colored precipitate; and/or

9) optionally, grinding and/or sieving, the colored precipitate.

48. A method to prepare a dispersible colorant comprising the steps:

1) dissolving the polysaccharide in water until the polysaccharide is completely dissolved;

2) adding the colorant to the solution of step 1) until the colorant is uniformly dispersed in the solution;

3) optionally adding a pH regulator to the solution of step 2);

4) forming the colored precipitate from the solution;

4) optionally drying the colored precipitate; and

5) optionally grinding and/or sieving the colored precipitate.

49. A method to prepare a dispersible colorant, comprising the steps:

1) preparing the aqueous solution of colorant and an aqueous solution of a metal salt, respectively;

2) dissolving the polysaccharide in the colorant solution prepared in step 1), for example, at a temperature of from about 0° C. to about 70° C., for about 1 minute to about 10 hours;

3) adding the aqueous solution of metal salt prepared in step 1) to the mixture of step 2) such that a colored precipitate forms, wherein, for example, the aqueous metal solution is added until the viscosity of the solution is similar or equal to water;

4) isolating the colored precipitate;

5) optionally, washing the colored precipitate with water and/or,

6) drying the colored precipitate, and/or

7) optionally grinding and/or sieving the colored precipitate.

50. A method to prepare a dispersible colorant, comprising the steps:

1) dissolving or dispersing a colorant in an aqueous solution to provide a colorant solution;

2) heating a polysaccharide in an aqueous solution until the polysaccharide is dissolved to provide a polysaccharide solution;

3) adding the colorant solution to the polysaccharide solution to form a colorant-polysaccharide mixture;

4) optionally maintaining heating the colorant-polysaccharide mixture for a period of time from about 10 minutes to about 24 hours;

5) cooling the colorant-polysaccharide mixture until a suspension or a precipitate is formed; and

6) isolating the colored precipitate or suspension material;

7) optionally, washing the colored precipitate or suspension material with water and/or,

8) drying the colored precipitate or suspension material, and/or

9) optionally grinding and/or sieving the colored precipitate or suspension material.

51. A method to prepare a dispersible colorant, comprising the steps:

1) heating a polysaccharide in an aqueous solution until the polysaccharide is dissolved to provide a polysaccharide solution;

2) dissolving or dispersing a colorant in an aqueous solution to provide a colorant solution;

3) adding the colorant solution to the polysaccharide solution to form a colorant-polysaccharide mixture;

4) optionally maintaining heating the colorant-polysaccharide mixture for a period of time from about 10 minutes to about 24 hours;

5) optionally cooling the colorant-polysaccharide mixture until a suspension or a precipitate is formed; and

6) isolating the colored precipitate or suspension material;

7) optionally, washing the colored precipitate or suspension material with water and/or,

8) drying the colored precipitate or suspension material, and/or

9) optionally grinding and/or sieving the colored precipitate or suspension material.

52. The method according to any one of paragraphs 46 through 51, wherein the polysaccharide is cross-linked starch.

53. The method according to any one of paragraphs 46 through 52, wherein the isolating step is spray drying to obtain a powdered product.

54. The method according to any one of paragraphs 46 through 53, wherein the colorant is encapsulated, gelatinized or coated by the polysaccharide to form the colored precipitate.

55. The method according to any one of paragraphs 46-50, wherein the temperature of the reaction medium after the addition of the polysaccharide is maintained from about 65° C. to about 85° C. for from about 5 minutes to about 24 hours.

56. Use of the disperse colorant according to any one of paragraphs 46 through 55 in applications wherein conventional colorants can be applied.

57. The use according to paragraph 56, wherein the disperse colorant is applied in the manufacture of food products, sweets, cosmetics, toys, and pharmaceutical products.

58. The use according to paragraph 57, wherein the food product is milk, butter, meat, candy, chocolates, ice cream, margarine, yogurt, fish or sugar.

59. The disperse colorant according to any of one or paragraphs 1 through 42 or 45 through 55, wherein the disperse colorant eliminates or reduces the smell associated with the colorant material.

60. A mixture of two or more disperse colorants according to any one of paragraphs 1 through 42 or 45 through 55, wherein the combination of the two or more disperse colorants provides any color within the visible spectrum.

61. A disperse colorant comprising:
a cellulose or a cellulose derivative and a colorant.

62. The disperse colorant according to paragraph 61, wherein the colorant is safflower yellow, sweet potato red, anthoblue, sodium copper chlorophyllin, gardenia blue, carminic acid, elderberry, red radish, beet root red, melanin, aronia and mixtures thereof.

63. The disperse colorant according to paragraph 61, wherein the colorant is curcumin, paprika red, lutein, β-carotene, lycopene and mixtures thereof.

64. The disperse colorant according to any of paragraphs 61 through 63, wherein the cellulose derivative is a cross-linked carboxymethylcellulose.

65. A disperse colorant comprising:
a starch or starch derivative and a colorant.

66. The disperse colorant according to paragraph 65, wherein the colorant is safflower yellow, sweet potato red, anthoblue, sodium copper chlorophyllin, gardenia blue, carminic acid, elderberry, red radish, beet root red, melanin, aronia and mixtures thereof.

67. The disperse colorant according to paragraph 65, wherein the colorant is curcumin, paprika red, lutein, β-carotene, lycopene and mixtures thereof.

68. The disperse colorant according to any of paragraphs 65 through 67, wherein the starch is a cross-linked starch.

### EXAMPLES

### Example 1

2 g of Safflower Yellow (WGFS 110121 E1%=140) was weighed and dissolved in 60 ml water at room temperature. 8.0 g crosslinked CMC (carboxymethylcellulose) was added to the safflower yellow solution with stirring. Citric acid was added until the pH reached 2.5 and the solution was stirred for 10 minutes to provide a solution. The solution was heated for 20 min in a water-bath at a temperature of 50° C. A colored precipitate was formed and was separated and washed twice with water. The precipitate was dried at 55° C. under vacuum to provide a final product.

### Example 2

The final product of example 1 was subjected to grinding, in order to form a powder, which was then sieved with a sieve of 240 mesh.

### Example 3

Following a process similar to example 1, combinations were prepared using Red Sweet Potato Color, gardenia blue, and sodium copper chlorophyllin, the conditions and results are summarized in Table 1.

TABLE 1

| | | | | Yield | |
|---|---|---|---|---|---|
| | | | | (after | |
| | | | | sieved | Color |
| Poly- | | | pH reg- | by 240 | of the |
| saccharide/g | Colorant/g | Water/mL | ulator/g | mesh)/g | product |
| crosslinked | Safflower | 60 | Citric | 5.2 | yellow |
| CMC, 8.0 | Yellow*, 2.0 | | acid, 0.8 | | |
| crosslinked | Red Sweet | 100 | Citric | 9.1 | red |
| CMC, 12.0 | Potato | | acid, 1.2 | | |
| | Color**, 3.0 | | | | |
| crosslinked | gardenia | 80 | Citric | 7.5 | blue |
| CMC, 10.5 | blue***, 0.7 | | acid, 1.0 | | |
| crosslinked | sodium copper | 80 | Citric | 4.2 | Deep |
| CMC, 5.0 | chlorophyllin, | | acid, 0.5 | | green |
| | 0.5 g | | | | |

*Safflower Yellow: WGFS110121 E1% = 140;
**Red Sweet Potato Color: WGFSP110426 E1% = 30;
***gardenia blue: E1% = 54

### Example 4

0.5 g of lutein was weighed and dissolved in 20 ml ethyl acetate at room temperature. 20 ml of water and 4 ml Triton X-100 ((C₁₄H₂₂O(C₂H₄O)ₙ) a nonionic surfactant which has a hydrophilic polyethylene oxide chain (on average 9.5 ethylene oxide units) and an aromatic hydrocarbon lipophilic or hydrophobic group. The hydrocarbon group is a 4-(1,1,3,3-tetramethylbutyl)-phenyl group) was added with stirring to form a mixture. 2.0 g crosslinked CMC was added to the mixture with stirring for 10 min. 0.2 g citric acid was then added with stirring until the crosslinked CMC was completely dissolved. The mixture was heated to 45° C. with stirring for 20 min. A precipitate was formed and separated by centrifugation and washed twice with water. The precipitate was dried at 55° C. under vacuum to provide a final product.

### Example 5

The final product of example 4 was subjected to grinding in order to form a powder, which was then sieved with a sieve of 240 mesh.

### Example 6

3.0 g CMC was weighed and dissolved in 50 ml water with stirring at room temperature until the CMC was completely dissolved. 1.0 g lutein was added into the solution with stirring until it was uniformly dispersed in the solution to provide a mixture. 1.0 g of citric acid was added to the mixture with stirring, until it was completely dissolved to provide a solution. The solution was dried, in order to obtain a precipitate, e.g., a colloid, as the final product.

### Example 7

The final product of example 6 was subjected to grinding in order to form a powder, which was then sieved with a sieve of 190 mesh.

### Example 8

Following a process similar to example 6, combinations were prepared using Red Sweet Potato Color, Safflower Yellow, and bilberry extract, the conditions and results were summarized in Table 2.

TABLE 2

| | | | | | Color |
|---|---|---|---|---|---|
| Poly- | | | pH reg- | | of the |
| saccharide/g | Colorant/g | Water/mL | ulator/g | Yield/g | product |
| CMC, 3.0 | Lutein, 1.0 | 50 | Citric | 3.7 | Orange |
| | | | acid, 1.0 | | |
| CMC, 2.0 | Red Sweet | 40 | Citric | 3.1 | Deep |
| | Potato Color, | | acid, 1.0 | | red |
| | 2.0 | | | | |
| CMC, 1.5 | Safflower | 60 | Citric | 3.6 | Light |
| | Yellow, 3.0 | | acid, 0.8 | | yellow |
| CMC, 1.2 | bilberry | 40 | Citric | 1.5 | Deep |
| | extract, 1.2 | | acid, 0.5 | | red |

The bilberry extract used herein is a product prepared from bilberry by sequential water extraction, refining, and spray drying, and is in the form of a water-soluble, dark red, powder.

### Example 9

0.5 g Red Sweet Potato Color was dissolved in 60 ml water at room temperature to obtain an aqueous solution. 5 g AlCl₃ was dissolved in 100 ml water at room temperature to obtain an aqueous solution. 2.0 g CMC was dissolved in the Red Sweet Potato Color solution at room temperature with stirring for 30 min. The AlCl₃ solution was added to the CMC-Red Sweet Potato Color mixutre dropwise with extensive stirring, such that a precipitate of CMC and Red Sweet Potato Color precipitates from the mixture. The aqueous solution of AlCl₃ was added while monitoring the viscosity of the reaction system, until the viscosity of the system was similar or equal to water. The precipitatie was washed with water, and dried at 60° C. under vacuum to provide a final product.

### Example 10

The final product of example 9 was subjected to grinding in order to form a powder, which was then sieved with a sieve of 190 mesh.

### Example 11

Following a process similar to example 9, combinations were prepared, the conditions and results were summarized in Table 3.

TABLE 3

| | | | | | Color |
|---|---|---|---|---|---|
| Poly- | | | pH reg- | | of the |
| saccharide/g | Colorant/g | Water/mL | ulator/g | Yield/g | product |
| CMC, 3.0 | Lutein, 1.0 | 50 | Citric | 3.7 | Orange |
| | | | acid, 1.0 | | |
| CMC, 2.0 | Red Sweet | 40 | Citric | 3.1 | Deep |
| | Potato Color, | | acid, 1.0 | | red |
| | 2.0 | | | | |

### Example 12

The red, yellow, and blue products prepared in Table 1 were compounded with each other, the conditions and results were summarized in Table 4.

TABLE 4

| Color 1 | Color 2 | Weight ratio | Color of the product |
|---|---|---|---|
| Yellow | Red | 1:1 | Orange |
| (crosslinked | (crosslinked | | |
| CMC + Safflower | CMC + Red | | |
| Yellow) | Sweet Potato | | |
| | Color) | | |
| Blue | Yellow | 1:1 | Green |
| (crosslinked | (crosslinked | | |
| CMC + | CMC + | | |
| gardenia | Safflower | | |
| blue) | Yellow) | | |
| Red | Blue | 1:1 | Purple |
| (crosslinked | (crosslinked | | |
| CMC + | CMC + | | |
| Red Sweet | gardenia | | |
| Potato Color) | blue) | | |

### Example 13

Red Sweet Potato Color, Safflower Yellow, and gardenia blue were dissolved in water, respectively, in order to obtain a solution of each colorant. The solutions are compounded with each other according to the weight ratio in Table 5. Crosslinked CMC was added with stirring; citric acid was added until the pH reached 2.5 and the solution was stirred. The solution was heated for 20 min in a water-bath at a temperature of 50° C. A colored precipitate was formed and was separated and washed twice with water. The precipitate was dried at 55° C. under vacuum to provide a final product.

The final products were subjected to grinding, in order to form a powder, which was then sieved with a sieve of 240 mesh.

| | | | | Yield | |
|---|---|---|---|---|---|
| | | | | (after | |
| | | | | sieved | Color |
| Crosslinked | | | Citric | by 240 | of the |
| CMC/g | Colorant/g | Water/mL | acid/g | mesh)/g | product |
| 30.0 | Red Sweet | 300 | 3.0 | 2.4 | Orange |
| | Potato | | | | |
| | Color:Saf- | | | | |
| | flower | | | | |
| | Yellow = | | | | |
| | 3.0:3.0 | | | | |
| 5.0 | Safflower | 50 | 0.5 | 4.2 | Green |
| | Yellow:gar- | | | | |
| | denia blue = | | | | |
| | 1.0:0.2 | | | | |
| 6.0 | Red Sweet | 60 | 0.6 | 4.3 | Purple |
| | Potato Color: | | | | |
| | gardenia blue = | | | | |
| | 1.0:0.5 | | | | |

### Example 14

### Thermal Stability Test

The yellow, red and blue product prepared in example 3 (hereafter referred to as Yellow Color, Red Color, and Blue Color) was covered by an edible oil and divided into two samples (i.e. a control sample and a test sample), respectively. The test samples were heated at 140° C. for 8 min under atmospheric conditions (oxygen present), cooled to room temperature, and then compared with the control samples. No observable color change was observed.

The result shows that the combinations described herein have good thermal stability.

### Example 15

### Light Stability Test

Test samples of Yellow Color, Red Color, and Blue Color were left for 7 days under indoor conditions (exposure to direct sunlight was avoided), and then compared with control samples. No observable color change was observed.

The result shows that the combinations described herein have good light stability.

### Example 15

### Use of the Combination Described Herein in the Manufacture of Color Clays

The samples of Yellow Color, Red Color and Blue Color, alone or in combination with each other, were added and kneaded into white clay substrates, until the samples were homogenously dispersed in the substrates. Many color clays with various colors were obtained by suitably compounding the 3 primary colors. The colors did not leak or “bleed” while the color clays were kneaded.

### Example 16

### Use of the Combination Described Herein in the Manufacture of Lipsticks

A substrate of lipstick material was melted at a temperature of 80° C. in a water-bath; Red Color was added into the melted substrate and the mixture was stirred until homogenous; the mixture was poured into a mold and cooled to obtain the lipstick. The Red Color was homogenously dispersed in the lipstick.

Results:

In comparison with conventional colorants and aluminum lakes, the dispersible colorants comprising a combination of a colorant and a polysaccharide described herein has the advantages of high light/thermal stability, high dispersibility, no bleeding, no toxicity, etc, and thus can be applied in the manufacture of food products, sweets, cosmetics, toys, pharmaceutical products, etc.

The above examples provide combinations of colorants and cellulose derivatives, such as crosslinked CMC. These can be applied in substrates such as foodstuffs, cosmetics, pharmaceuticals, nutraceuticals, cosmetics, etc.

The following examples provide combinations of colorants and starch derivatives, such as crosslinked starch. These can also be applied in foodstuffs, but can also be applied in other substrates, such as pharmaceuticals, nutraceuticals, cosmetics, etc.

The following examples were spray dried under the following parameters: inlet temperature: about 160° C. to about 240° C., more particularly 190° C. to about 200° C.; outlet temperature 80° C. to about 120° C., more particularly 90° C. to about 100° C.

In the following examples, a precipitate or a suspension was formed as the product prior to spray drying. This should not be limiting as a product could form as a solution and be spray dried as well.

Raw Materials:

Crosslinked starch: acetylated distarch adipate (acetylation value=1.5-2.1, from Roquette, Italy)

### Example 1

### Preparation of Safflower Yellow Powder Colorant Combination (ColorDispersible 2Y-01; CD-Yellow

1. 600 g safflower yellow (from Wuhan Green Food Biological Engineering Co., Ltd.) (nominal E1%=144.2, measured E1%=146.2, lot: WGFS121223) was dissolved in 10 L purified water to obtain a safflower yellow solution;

2. 320 L purified water was added into a 500 L reaction tank, the system was heated rapidly to 60° C.-65° C. by steam;

3. 8 kg crosslinked starch was homogenously dispersed in 20 L purified water to obtain a suspension, and the suspension was added into the reaction tank using a vacuum pump;

4. the reaction tank was slowly heated to 70° C. by steam for gelatinization, the temperature was held at 70° C. for about 20-30 min, and then was cooled by cooling water to below 55° C.;

5. the safflower yellow solution was added into the reaction tank using a vacuum pump;

6. the cooling water was removed when the temperature reached 48° C.-50° C., and the temperature was held for about 40 min-60 min;

7. the product was discharged as a suspension, spray dried, and was pulverized until the particle size <50 μm.

Mass of the product: 5.48 kg

Color value: 11.6

Mass yield: 63.72%

The dispersible colorant was applied to pasta and was found to not bleed. No diffusion of the colorant combination occurred at the boundaries of application.

### Example 2

### Preparation of Sweet Potato Red Powder Colorant Combination (ColorDispersible 2R-03; CD-Red)

1. 1.6 kg sweet potato red (from Wuhan Green Food Biological Engineering Co., Ltd.) (nominal E1%=31.8, measured E1%=33.4, lot: WGFSP121222) and 384 g citric acid was dissolved in 10 L purified water to obtain a sweet potato red solution;

2. 340 L purified water was added into a 500 L reaction tank, the system was heated rapidly to 60° C.-65° C. by steam;

3. 8 kg crosslinked starch was homogenously dispersed in 20 L purified water (hot water from the reaction tank) to obtain a suspension, and the suspension was added into the reaction tank using a vacuum pump;

4. the reaction tank was slowly heated to 70° C. by steam for gelatinization, the temperature was held at 70° C. for about 20-30 min, and then was cooled by cooling water to below 55° C.;

5. the sweet potato red solution was added into the reaction tank using the vacuum pump;

6. the cooling water was removed when the temperature reached 48° C.-50° C., and the temperature was held for about 40 min-60 min;

7. the product was discharged as a suspension, spray dried, and pulverized until the particle size <50 m.

Mass of the product: 7.65 kg

Color value: 6.9

Mass yield: 76.62%

The colorant combination was applied to pasta and was found to not bleed. No diffusion of the colorant combination occurred at the boundaries of application.

### Example 3

### Preparation of Anthoblue Powder Colorant Combination (CD-AnthoBlue)

1. 1.29 kg Anthoblue (acetylated anthocyanin purified from red cabbage, from Ningbo Green-Health Pharmaceutical Co., Ltd., nominal E1%=51.9, measured E1%=53.8) and 327 gAlCl₃.6H₂O (180.76 g) was dissolved in 10 L purified water to obtain a red cabbage red solution;

2. 340 L purified water was added into a 500 L reaction tank, the system was heated rapidly to 60° C.-65° C. by steam;

3. 8 kg crosslinked starch was homogenously dispersed in 20 L purified water (hot water from the reaction tank) to obtain a suspension, and the suspension was added into the reaction tank using a vacuum pump;

4. the reaction tank was slowly heated to 70° C. by steam for gelatinization, the temperature was held at 70° C. for about 20-30 min, and then was cooled by cooling water to below 55° C.;

5. the red cabbage red solution was added into the reaction tank using the vacuum pump, and the pH value of the system was adjusted to 4.0-4.5 by NaOH;

6. the cooling water was removed when the temperature reached 48° C.-50° C., and the temperature was held for about 40 min-60 min;

7. the product was discharged as a suspension, spray dried, and pulverized until the particle size <50 μm.

Mass of the product: 7.86 kg

Color value: 10.6

Mass yield: 81.86%

The colorant combination was applied to pasta and was found to not bleed. No diffusion of the colorant combination occurred at the boundaries of application.

The application of conventional red cabbage red colorant is quite limited due to its unpleasant smell. The inventors surprisingly find that, the combination described herein removes the unpleasant smell of red cabbage red colorant advantageously.

### Example 4

### Preparation of Sodium Copper Chlorophyllin Powder Colorant Combination (CD-SCC)

1. 352 g copper sodium chlorophyll (From Shandong Guangtongbao Pharmaceuticals Co., Ltd.) (measured E1%=490.4 g) was dissolved in 10 L purified water to obtain a copper sodium chlorophyll solution;

2. 340 L purified water was added into a 500 L reaction tank, the system was heated rapidly to 60° C.-65° C. by steam;

3. 8 kg crosslinked starch was homogenously dispersed in 20 L purified water (hot water from the reaction tank) to obtain a suspension, and the suspension was added into the reaction tank using a vacuum pump;

4. the reaction tank was slowly heated to 70° C. by steam for gelatinization, the temperature was held at 70° C. for about 20-30 min, and then was cooled (by cooling water) to below 55° C.;

5. the copper sodium chlorophyll was added into the reaction tank using the vacuum pump, and the pH value of the system was adjusted to 3.5-4.0 by HCl;

6. the cooling water was removed when the temperature reached 48° C.-50° C., and the temperature was held for about 40 min-60 min;

7. the product was discharged as a suspension, spray dried, and pulverized until the particle size <50 μm.

Mass of the product: 6.65 kg

Color value: 20.3

Material balance: yield of color value=56.66%

Mass yield: 79.62%

The colorant combination was applied to pasta and was found to not bleed. No diffusion of the colorant combination occurred at the boundaries of application.

### Example 5

### Preparation of Curcumin Powder Colorant Combination

1. 5 g crosslinked starch was added into 50 ml water,

2. the system was heated to 68-70° C. for gelatinization, the temperature was held for about 20 min-30 min, and then was cooled by cooling water to 48° C.-50° C.;

3. 0.5 g curcumin (crystal size <50 μm, E1%=1465, determined in 50% ethanol aqueous solution, from Ningbo Traditional Chinese Pharmaceuticals Co., Ltd.) was added into the system and stirred until homogenous, and the temperature was held for about 40 min—60 min;

4. the product was discharged as a suspension, spray dried, and pulverized until the particle size <50 m.

Mass of the product: 3.5 g

Color value: 135

Mass yield: 63.6%

The colorant combination was applied to pasta and was found to not bleed. No diffusion of the colorant combination occurred at the boundaries of application.

### Example 6

### Preparation of Paprika Red Colorant Combination

1. 2 g crosslinked starch was dispersed into 20 ml purified water;

2. the system was heated to 68-70° C. in water bath for gelatinization, the temperature was held for about 20 min-30 min, and then was cooled by cooling water to below 50° C.;

3. 0.2 g paprika red colorant was added with stirring, and the system was continuously stirred at room temperature until homogeneous;

4. a suitable amount of water was added into the system in order to reduce the viscosity;

5. the system was spray dried to provide a orange (or yellow) product.

The colorant combination was applied to pasta and was found to not bleed. No diffusion of the colorant combination occurred at the boundaries of application.

The application of conventional paprika red colorant is quite limited due to its unpleasant smell. The inventors surprisingly find that, the combination described herein advantageously eliminates the unpleasant smell of paprika red colorant.

### Example 7

### Preparation of Gardenia Blue Colorant Combination

1. 5 g crosslinked starch was dispersed into 50 ml purified water;

2. the system was heated to 68-70° C. in water bath for gelatinization, the temperature was held for about 20 min-30 min, and then was cooled by cooling water to below 50° C.;

3. 0.2 g gardenia blue colorant was added with stirring, and the system was continuously stirred at room temperature until homogeneous;

4. a suitable amount of water was added into the system in order to reduce the viscosity;

5. the system was spray dried to provide a blue product.

The colorant combination was applied to pasta and was found to not bleed. No diffusion of the colorant combination occurred at the boundaries of application.

### Example 8

### Preparation of Carminic Acid Colorant Combintion

1. 10 g crosslinked starch was dispersed into 50 ml purified water;

2. the system was heated to 68-70° C. in water bath for gelatinization, the temperature was held for about 20 min-30 min, and then was cooled by cooling water to below 50° C.;

3. 0.5 g carminic acid colorant was added with stirring, and the system was continuously stirred at room temperature until homogeneous:

4. a suitable amount of water was added into the system in order to reduce the viscosity;

5. the system was spray dried to provide a red product.

The colorant combination was applied to pasta and was found to not bleed. No diffusion of the colorant combination occurred at the boundaries of application.

### Example 9

### Preparation of Elderberry Extract Combination

1. 10 g crosslinked starch was dispersed into 50 ml purified water;

2. the system was heated to 68-70° C. in water bath for gelatinization, the temperature was held for about 20 min-30 min, and then was cooled by cooling water to below 50° C.;

3. 1.0 g elderberry extract was added with stirring, and the system was continuously stirred at room temperature until homogeneous;

4. a suitable amount of water was added into the system in order to reduce the viscosity;

5. the system was spray dried to provide a deep red product.

The colorant combination was applied to pasta and was found to not bleed. No diffusion of the colorant combination occurred at the boundaries of application.

### Example 10

### Preparation of Lutein Combination

1. 5 g crosslinked starch was dispersed into 50 ml purified water;

2. the system was heated to 68-70° C. in water bath for gelatinization, the temperature was held for about 20 min-30 min, and then was cooled by cooling water to below 50° C.;

3. 0.5 g lutein (crystal size <50 μm) was added with stirring, the system was continuously stirred at room temperature until homogeneous, and the temperature was held for 40-60 min;

4. the system was spray dried, and pulverized to below 50 μm, to provide the product.

The colorant combination was applied to pasta and was found to not bleed. No diffusion of the colorant combination occurred at the boundaries of application.

### Example 11

### Preparation of Red Radish Combination

1. 5 g crosslinked starch was dispersed into 50 ml purified water;

2. the system was heated to 68-70° C. in water bath for gelatinization, the temperature was held for about 20 min-30 min, and then was cooled by cooling water to below 50° C.;

3. 0.5 g red radish colorant was added with stirring, and the system was continuously stirred at room temperature until homogeneous;

4. a suitable amount of water was added into the system in order to reduce the viscosity;

5. the system was spray dried to provide the product.

The colorant combination was applied to pasta and was found to not bleed. No diffusion of the colorant combination occurred at the boundaries of application.

The application of conventional red radish colorant is quite limited due to its unpleasant smell. The inventors surprisingly find that, the colorant combination described herein advantageously eliminates the unpleasant smell of red radish colorant.

### Example 12

### Preparation of beet root red combination

1.5 g crosslinked starch was dispersed into 50 ml purified water;

2. the system was heated to 68-70° C. in water bath for gelatinization, the temperature was held for about 20 min-30 min, and then was cooled by cooling water to below 50° C.;

3. 0.5 g beet root red colorant was added with stirring, and the system was continuously stirred at room temperature until homogeneous;

4. a suitable amount of water was added into the system in order to reduce the viscosity;

5. the system was spray dried to provide the product.

The colorant combination was applied to pasta and was found to not bleed. No diffusion of the colorant combination occurred at the boundaries of application.

### Example 13

### Preparation of Melanin Combination

1.5 g crosslinked starch was dispersed into 50 ml purified water;

2. the system was heated to 68-70° C. in water bath for gelatinization, the temperature was held for about 20 min-30 min, and then was cooled by cooling water to below 50° C.;

3. 0.5 g melanin was added with stirring, and the system was continuously stirred at room temperature until homogeneous;

4. a suitable amount of water was added into the system in order to reduce the viscosity;

5. the system was spray dried to provide the product.

The colorant combination was applied to pasta and was found to not bleed. No diffusion of the colorant combination occurred at the boundaries of application.

### Example 14

### Preparation of β-Carotene Composition

1.5 g crosslinked starch was dispersed into 50 ml purified water;

2. the system was heated to 68-70° C. in water bath for gelatinization, the temperature was held for about 20 min-30 min, and then was cooled by cooling water to below 50° C.;

3. 0.5 g β-carotene carotene was added with stirring, and the system was continuously stirred at room temperature until homogeneous;

4. a suitable amount of water was added into the system in order to reduce the viscosity;

5. the system was spray dried to provide the product.

The colorant combination was applied to pasta and was found to not bleed. No diffusion of the colorant combination occurred at the boundaries of application.

Notes:

1. Advantageous gelatinization temperature range: 65° C.-85° C.

2. Classification of the colors in the examples:

Water soluble color: safflower yellow; sweet potato red; Anthoblue; sodium copper chlorophyllin; gardenia blue; carminic acid; elderberry; red radish; beet root red; and melanin (alkaline water soluble).

Oil soluble color: curcumin; paprika red; lutein; and β-carotene.

Comparison Between the Colorants According to Embodiments Described Herein and Conventional Colorants

Abbreviations

CD-SP: ColorDispersible 2R-03 Powder (Sweet Potato) described above.

CD-S: ColorDispersible 2Y-01 Powder (Safflower Yellow) described above.

SP: (conventional) Sweet Potato Red Powder

SP-S: (conventional) Sweet Potato Red aqueous solution

AB: Anthoblue (acetylated anthocyanin purified from red cabbage, from Ningbo Green-Health Pharmaceutical Co., Ltd.) powder

AB-S: AnthoBlue aqueous solution

CD-AB: CD-AnthoBlue

### Example 1

### Comparison Between CD-SP and Conventional SP in Peanut Oil

5 ml Peanut oil was mixed with 4 mg SP or 10 mg CD-SP

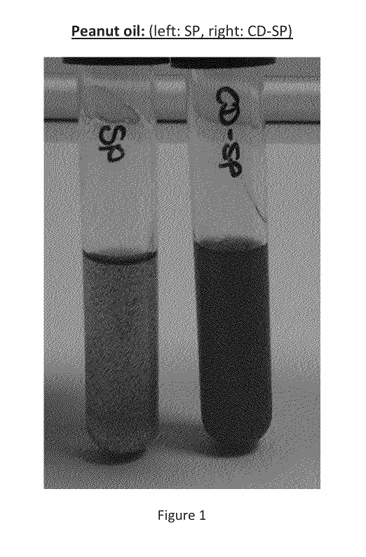

As can be seen from FIG. 1, CD-SP is well dispersed in peanut oil, while SP was not.

### Example 2

### Comparison Between CD-SP/CD-S and Conventional Synthetic and Natural Colorants in Sunflower Oil

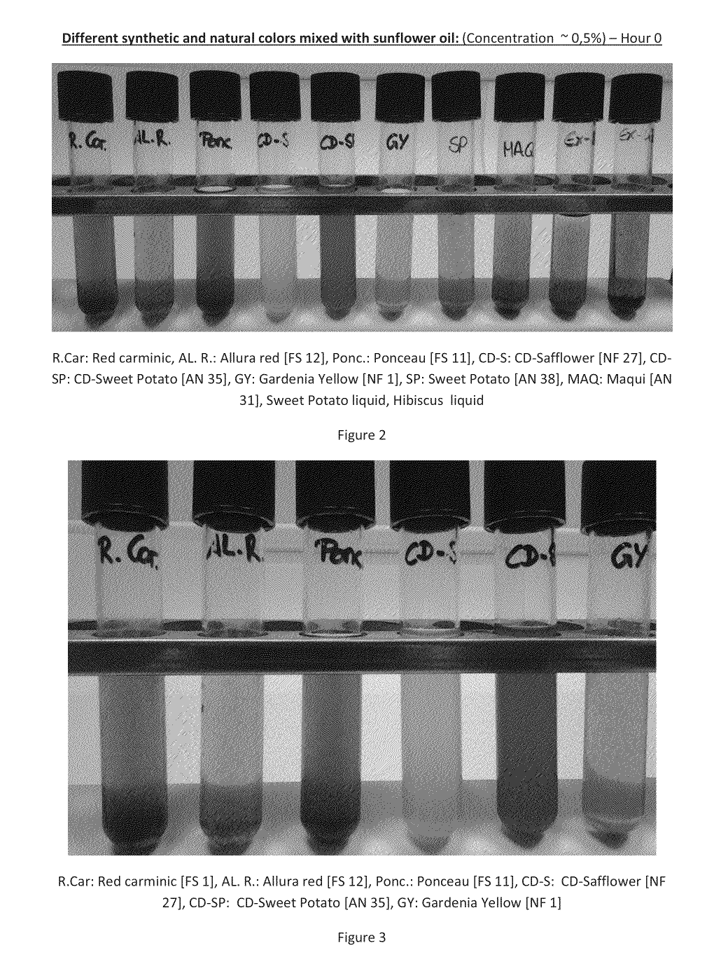

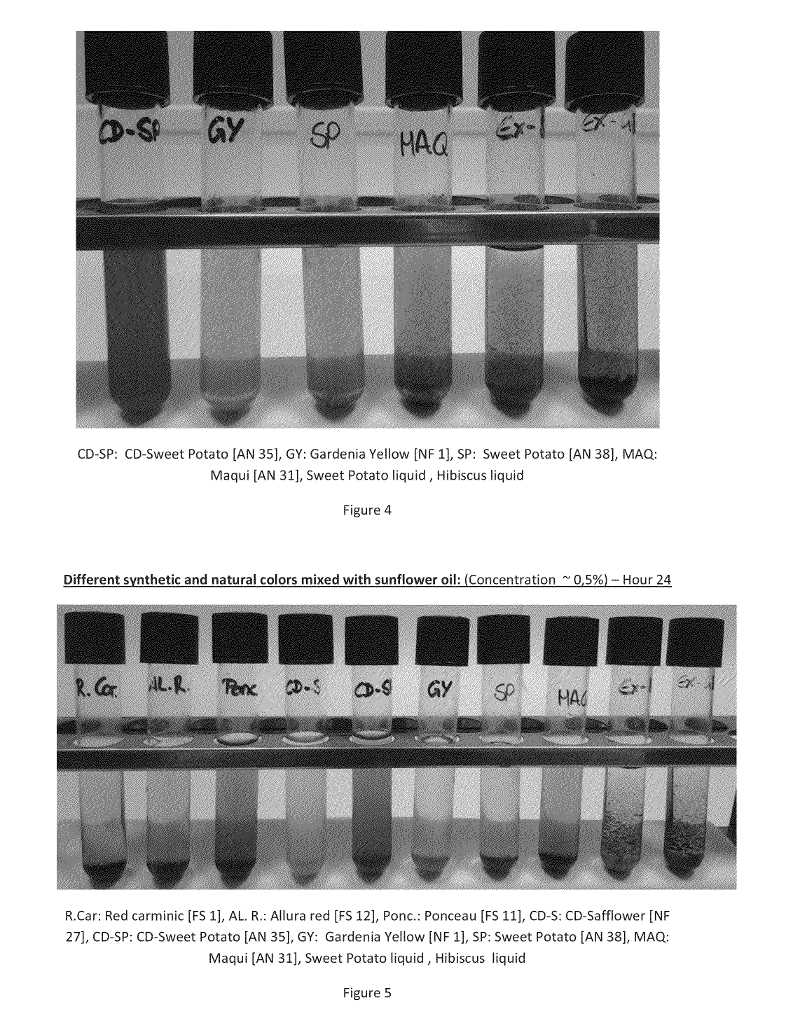

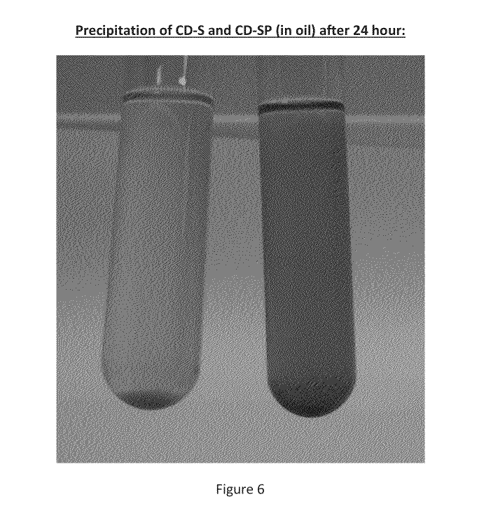

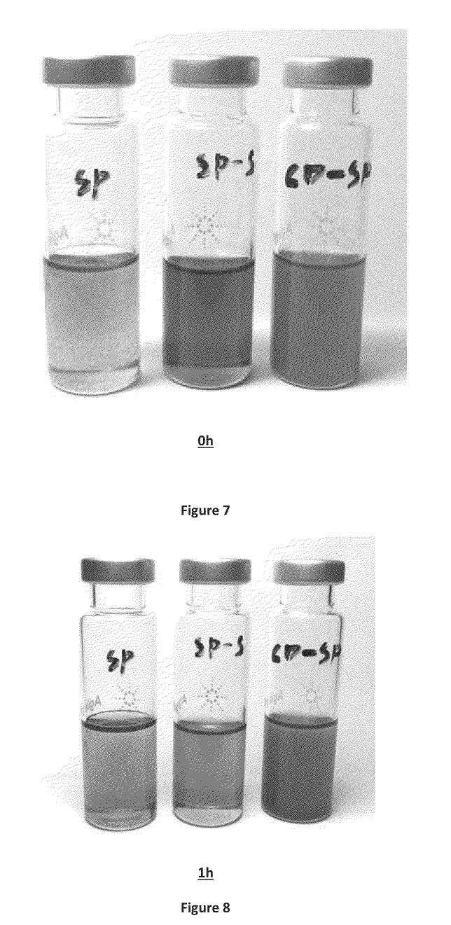

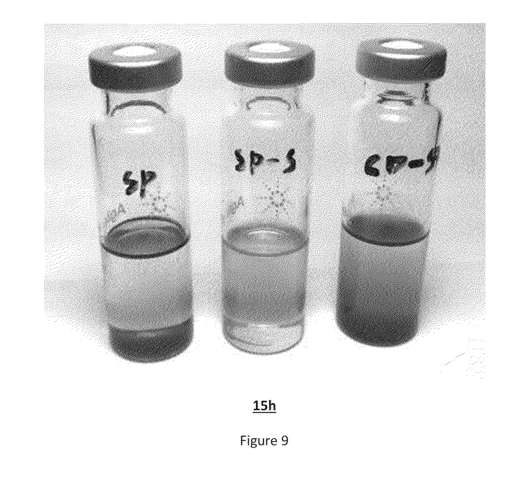

Different conventional synthetic and natural colorants, as well as CD-SP and CD-S described above, was mixed with sunflower oil and was allowed to stand for 24 h, respectively. As shown in FIG. 2-FIG. 4, at 0 h, both CD-SP and CD-S were well dispersed in sunflower oil and homogenous systems were obtained, while conventional synthetic and natural colors cannot. FIGS. 3 and 4 are enlarged views of FIG. 2. As shown in FIG. 5 and FIG. 6, after 24 h, both CD-SP and CD-S were still well dispersed in sunflower oil, with little precipitation and the systems keep their original color, while conventional synthetic and natural colors were substantially precipitated from sunflower oil and the systems became transparent (i.e. conventional synthetic and natural colorants cannot be applied in such oil systems).

The results showed that CD-SP and CD-S described herein has significantly higher dispersibility and stability in oil system than conventional synthetic and natural colorants.

### Example 3

### Comparison Between CD-SP and Conventional SP/SP-S in PEG (400)

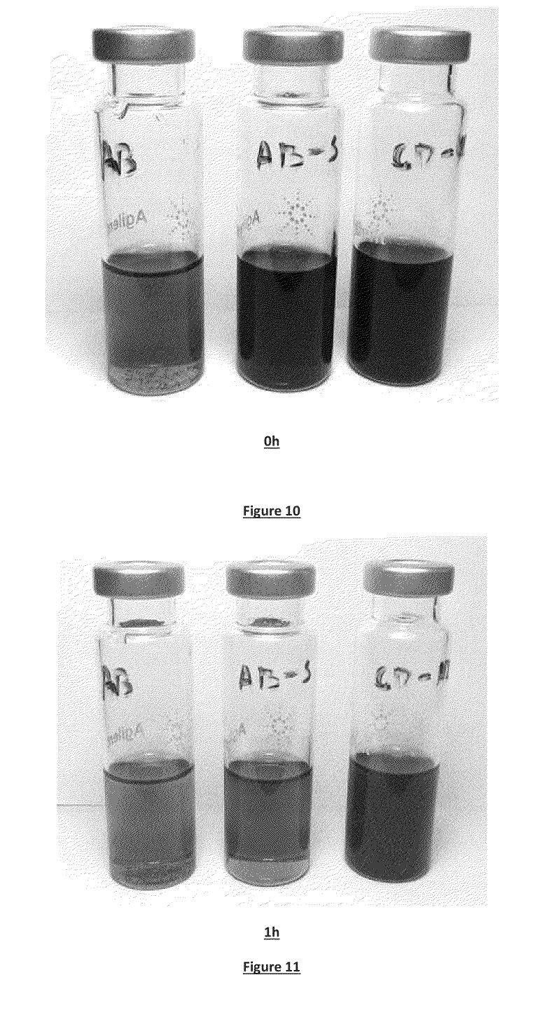

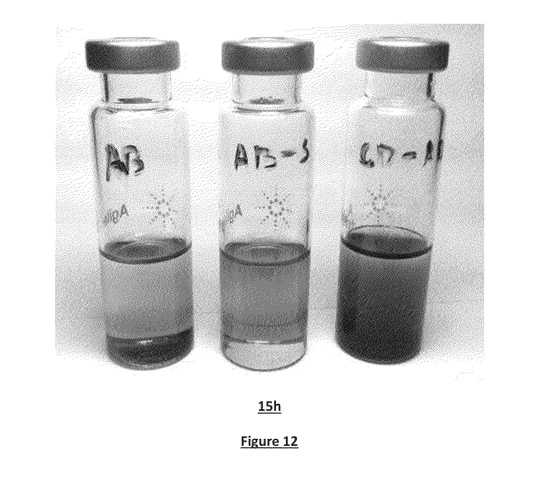

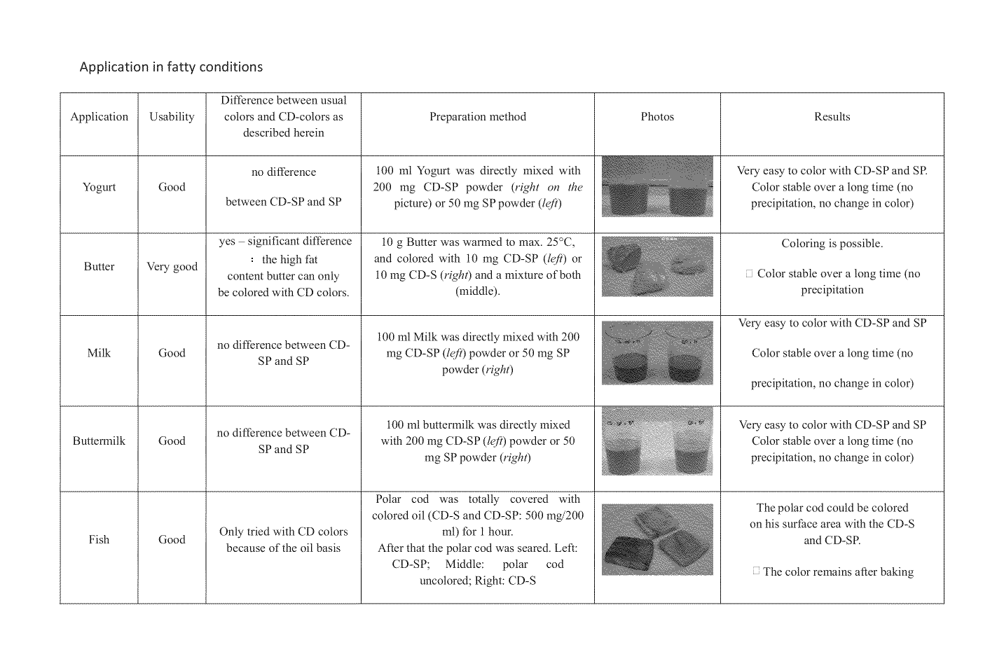

As can be seen from FIGS. 7-9, at 0 h (FIG. 7), a homogeneous colored system can be obtained with SP-S and CD-SP, but cannot be obtained with SP; after 1 h, the colorant precipitated from the SP-S system, and the color of the SP-S system became lighter, while CD-SP system kept its original color (FIG. 8); after 15 h, the colorants were substantially precipitated from the SP system and the SP-S system, and such systems became colorless, however, little colorant precipitated from the CD-SP colorant (FIG. 9). The result showed that CD-SP colorant has significantly higher dispersability and stability in a PEG (400) system. Conventional colorants are not applicable in such a system, but the colorant described herein is applicable.

### Example 4

### Comparison Between CD-AB, Conventional AB, and AB-S in PEG 400

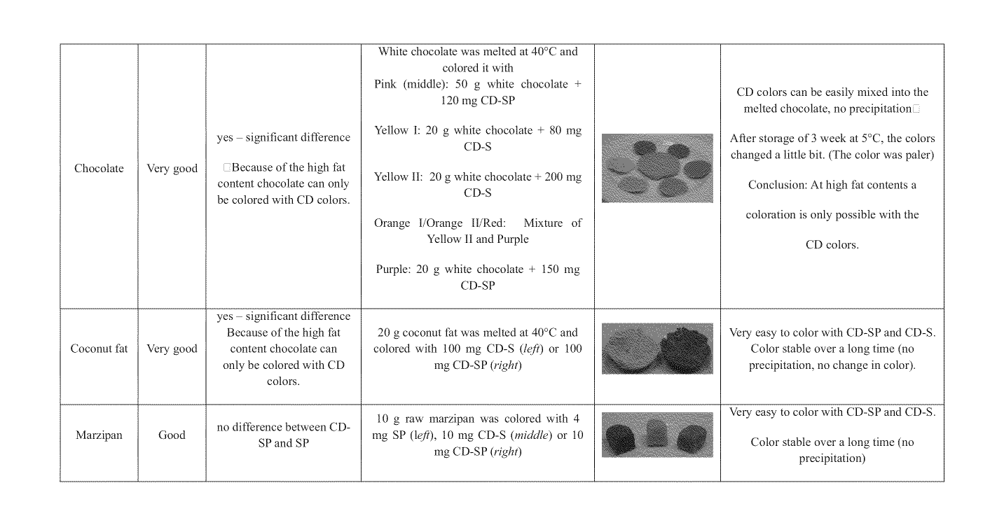

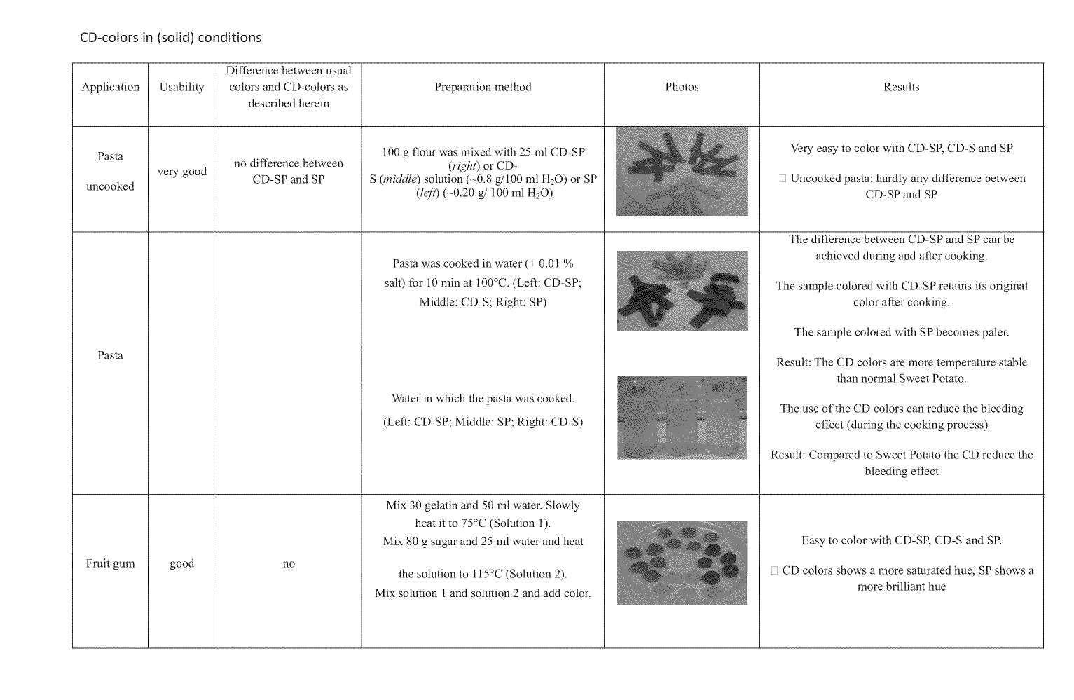

As can be seen from FIGS. 10-12, at 0 h (FIG. 10), a homogeneous colored system can be obtained with AB-S and CD-AB, but cannot be obtained with AB; after 1 h, the colorant precipitated from the AB-S system, and the color of the SP-S system changed, while CD-AB system kept its original color (FIG. 111); after 15 h, the colorants were substantially precipitated from the ABsystem and the AB-S system, and the color of such systems changed significantly, however, little colorant precipitated from the CD-AB colorant (FIG. 12). The results showed that CD-AB colorant has significantly higher dispersability and stability in a PEG (400) system. Conventional colorants are not applicable in such system, but the colorant as described herein is applicable.

Application of compositions described herein in fatty conditions

Reagents

| | | Abbrevia- |
|---|---|---|
| Product Name | Producer | tion |
| ColorDispersible 2R-03 powder (Sweet | Described | CD-SP |
| Potato) | herein | |
| ColorDispersible 2Y-01 powder | Described | CD-S |
| (Safflower Yellow) | herein | |
| Sweet Potato Red Powder | Wuhan Green | SP |
| | Food | |
| | Biological | |
| | Engineering Co., | |
| | Ltd. | |
| ColorDispersible Anthoblue Powder F | Described | CD-AB |
| | herein | |
| Quality first Polar cod | Billa Austria | Polar cod |
| S-Budget Vanilla ice cream (10 g fat/ | Spar Austria | Ice cream |
| 100 g) | | |
| Spar Mayonnaise (50% fat) (=salad | Spar Austria | Salad |
| cream) | | cream |
| Ceres (100% coconut fat) | VFI Wels | Coconut |
| | | fat |
| Schärdinger creamy nature yogurt | Berglandmilch, | yogurt |
| (3.6% fat) | Wels | |
| Spar cream (36% fat) | Berglandmilch, | cream |
| | Wels | |
| Wiener sugar (crystallized sugar) | Agrana, Wien | sugar |
| Austrian butter (out of pasteurized milk) | Pinzgauer Milch | butter |
| (mind. 82% fat) | | |
| Zentis Marzipan | Zentis GmbHCo | marzipan |
| | KG | |
| Clever flour (Type W 480) - wheat | Delikatessa | flour |
| | GmbH | |
| Haas Instant gelatin (mixture of gelatin | Ed. Haas | gelatine |
| and glucose syrup) | Austria | |
| Ja natürlich! Sunflower oil | Ja natürlich! | Sunflower |
| | | oil |
| Milka White chocolade | Milka | chocolate |

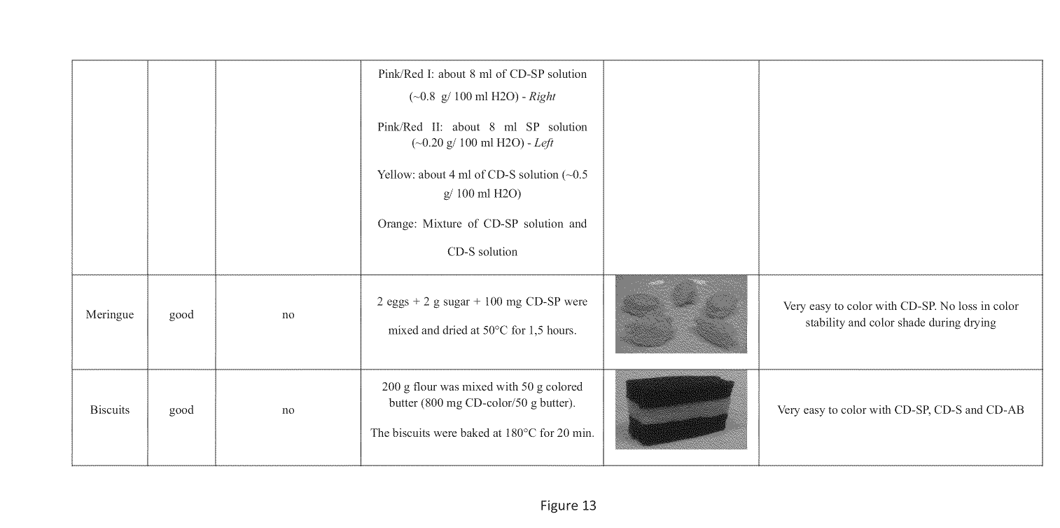

FIG. 13 provides uses of various embodiments described herein in various foodstuffs as noted above. Some observations that can be drawn from the use in foodstuffs include that CD-colors (for example in pasta) the CD-colors are heat resistant even at elevated temperatures and they do not degrade to any significance; the disadvantage of bleeding can be reduced, for example: cooking of pasta does not appreciably bleed into the water; and that CD-colors can be used in fatty (oleophobic/lipophobic) formulations and in solid formulations.

### Example 5

### Dispersing CD-SP is Water with Heat to Provide a Swelled Material

50 ml distilled water and 900 mg of CD-SP were heated in water for 3 minutes at 100° C. to produce a viscous solution. The viscous solution was then dried at room temperature for 48 hours to afford a red powder. The resultant red powder was dispersible in or an oil.

Although the present invention has been described with reference to preferred embodiments, persons skilled in the art will recognize that changes may be made in form and detail without departing from the spirit and scope of the invention. All references cited throughout the specification, including those in the background, are incorporated herein in their entirety. Those skilled in the art will recognize, or be able to ascertain, using no more than routine experimentation, many equivalents to specific embodiments of the invention described specifically herein. Such equivalents are intended to be encompassed in the scope of the following claims.

What is claimed is:

1. A dispersible colorant comprising a combination of a colorant and a polysaccharide, wherein the weight ratio of total colorant to total polysaccharide is in the range of 5000:1 to 1:5000.

2. The dispersible colorant according to claim 1, wherein the polysaccharide is cellulose, a cellulose derivative or a cross-linked carboxymethylcellulose.

3. The dispersible colorant according to claim 1, wherein the colorant is a natural colorant.

4. The dispersible colorant according to claim 1, wherein the colorant is a natural edible colorant.

5. The dispersible colorant according to claim 1, wherein the colorant is safflower yellow, sweet potato red, anthoblue, sodium copper chlorophyllin, gardenia blue, carminic acid, elderberry, red radish, beet root red, melanin, aronia and mixtures thereof.

6. The dispersible colorant according to claim 2, wherein the colorant is safflower yellow, sweet potato red, anthoblue, sodium copper chlorophyllin, gardenia blue, carminic acid, elderberry, red radish, beet root red, melanin, aronia and mixtures thereof.

7. The disperse colorant according to claim 1, wherein the colorant is curcumin, paprika red, lutein, J-carotene, lycopene and mixtures thereof.

8. The disperse colorant according to claim 2, wherein the colorant is curcumin, paprika red, lutein, β-carotene, lycopene and mixtures thereof.

9. The dispersible colorant according to claim 1, wherein the polysaccharide is starch, a starch derivative or a cross-linked starch.

10. The dispersible colorant according to claim 9, wherein the colorant is a natural colorant.

11. The dispersible colorant according to claim 9, wherein the colorant is a natural edible colorant.

12. The dispersible colorant according to claim 9, wherein the colorant is safflower yellow, sweet potato red, anthoblue, sodium copper chlorophyllin, gardenia blue, carminic acid, elderberry, red radish, beet root red, melanin, aronia and mixtures thereof.

13. The disperse colorant according to claim 9, wherein the colorant is curcumin, paprika red, lutein, β-carotene, lycopene and mixtures thereof.

14. The use of a disperse colorant according to claim 1, wherein the dispersible colorant is applied in the manufacture of food products, sweets, chocolates, cosmetics, toys, beverages, nutraceuticals, or pharmaceutical products.

15. A method to impart a color to a food product, a sweet, a chocolate, a cosmetic, a toy material or a pharmaceutical product comprising the step of adding a dispersible colorant according to claim 1 to the food product, sweet, chocolate, cosmetic, toy material or pharmaceutical product.

16. A method to prepare a dispersible colorant, comprising the steps:
1) dissolving a water-soluble colorant in water to form a solution, wherein the colorant is present from an amount greater than zero to saturation in the water,
2) adding a polysaccharide to the solution prepared in step 1) to form a mixture;
3) optionally adding a pH regulator to the mixture of step 2) until the pH of the mixture reaches a pH of about 2 to about 8.5 to form a mixture;
4) heating the mixture obtained from step 3), followed by optionally cooling the mixture until a solution, colored precipitate or suspension is formed; and
5) collecting the dispersible colorant.

17. The method of claim 16, further comprising the step of subjecting the solution, colored precipitate or suspension to spray drying to obtain a powdered dispersible colorant.

18. A method to prepare a dispersible colorant, comprising the steps:
1) dissolving an oil-soluble colorant in an organic solvent or a mixture of organic solvents to form a mixture, wherein the colorant is present from an amount greater than zero to saturation in the organic solvent(s);
2) adding a surfactant to the mixture of step 1), optionally with water to form a mixture;
3) adding a polysaccharide to the mixture of step 2) to form a mixture;
4) optionally adding a pH regulator to the mixture of step 3) until the pH of the mixture reaches a pH of about 2 to about 8.5 to form a mixture;
(5) heating the mixture of step 4) followed optionally by cooling the mixture until a solution, colored precipitate or suspension is formed; and
(6) collecting the dispersible colorant.

19. The method of claim 16, further comprising the step of subjecting the solution, colored precipitate or suspension to spray drying to obtain a powdered dispersible colorant.

20. The method of claim 16, further comprising the steps;
dispersing the dispersible colorant in a solvent to form a mixture;
heating the mixture between 60° C. and 120° C. to treat the dispersible colorant; and
cooling the mixture to provide a heat treated dispersible colorant.

21. The method of claim 20, wherein the solvent is water.

22. The method of claim 16, further comprising the step of heating the dispersible colorant to a temperature below the melting or degradation point and cooling the dispersible colorant to provide a heat treated dispersible colorant.

23. The method of claim 18, further comprising the steps;
dispersing the dispersible colorant in a solvent to form a mixture;
heating the mixture between 60° C. and 120° C. to treat the dispersible colorant; and
cooling the mixture to provide a heat treated dispersible colorant.

24. The method of claim 23, wherein the solvent is water.

25. The method of claim 18, further comprising the step of heating the dispersible colorant to a temperature below the melting or degradation point and cooling the dispersible colorant to provide a heat treated dispersible colorant.

26. A method to prepare a dispersible colorant, comprising the steps:
1) dispersing a starch in water to obtain a suspension;
2) heating the aqueous suspension to gelatinize the starch;
3) adding an oil-soluble colorant or an aqueous solution of water-soluble colorant into the gelatinized starch to provide a mixture; and
4) optionally adjusting the pH of the mixture to afford a dispersible colorant mixture.

27. The process of claim 26, further comprising the step of isolating the dispersible colorant mixture.

28. The process of claim 27, wherein the isolation step is by spray drying.

29. The process of claim 26, wherein the starch is a cross-linked starch.

30. The process of claim 26, wherein the aqueous suspension is heated to a temperature below 100° C.

31. The process of claim 30, wherein the temperature is between about 60° C. and about 80° C.

32. The process of claim 26, wherein the colorant is safflower yellow, sweet potato red, anthoblue, sodium copper chlorophyllin, gardenia blue, carminic acid, elderberry, red radish, beet root red, melanin, aronia, curcumin, paprika red, lutein, β-carotene, lycopene, and mixtures thereof

33. The method of claim 26, further comprising the steps:
dispersing the dispersible colorant in a solvent to form a mixture;
heating the mixture between 60° C. and 120° C. to treat the dispersible colorant; and
cooling the mixture to provide a heat treated dispersible colorant.

34. The method of claim 33, wherein the solvent is water.

35. The method of claim 26, further comprising the step of heating the dispersible colorant to a temperature below the melting or degradation point and cooling the dispersible colorant to provide a heat treated dispersible colorant.

36. A heat treated dispersible colorant comprising a starch, a starch derivative or a crosslinked starch and a colorant.

37. The heat treated dispersible colorant according to claim 36, wherein the colorant is safflower yellow, sweet potato red, anthoblue, sodium copper chlorophyllin, gardenia blue, carminic acid, elderberry, red radish, beet root red, melanin, aronia and mixtures thereof.

38. The heat treated dispersible colorant according to claim 36, wherein the colorant is curcumin, paprika red, lutein, J-carotene, lycopene and mixtures thereof.

39. Use of the heat treated dispersible colorant according to claim 36, wherein the dispersible colorant is applied in the manufacture of food products, sweets, nutraceuticals, beverages, chocolates, cosmetics, toys, or pharmaceutical products.

40. The dispersible colorant of claim 14, wherein the nutraceutical or pharmaceutical product is in the form of a tablet, capsule, powder, solution, injectable or granulated.

41. The heat treated dispersible colorant of claim 39, wherein the nutraceutical or pharmaceutical product is in the form of a tablet, capsule, powder, solution, injectable or granulated.
